SEQUENCE LISTING (9 sequences)

SEQ ID NO: 1 - protein, 8 aa, Artificial
  Synthetic peptide
  misc_feature (2)..(5): Xaa can be any naturally occurring amino acid
GXXXXGKS

SEQ ID NO: 2 - protein, 217 aa, Artificial
  Synthetic peptide
  misc_feature (14)..(14): Xaa can be any naturally occurring amino acid
  misc_feature (66)..(66): Xaa can be any naturally occurring amino acid
  misc_feature (68)..(69): Xaa can be any naturally occurring amino acid
  misc_feature (71)..(73): Xaa can be any naturally occurring amino acid
  misc_feature (75)..(76): Xaa can be any naturally occurring amino acid
  misc_feature (78)..(78): Xaa can be any naturally occurring amino acid
  misc_feature (83)..(84): Xaa can be any naturally occurring amino acid
  misc_feature (86)..(86): Xaa can be any naturally occurring amino acid
  misc_feature (88)..(88): Xaa can be any naturally occurring amino acid
  misc_feature (90)..(90): Xaa can be any naturally occurring amino acid
  misc_feature (92)..(95): Xaa can be any naturally occurring amino acid
  misc_feature (98)..(104): Xaa can be any naturally occurring amino acid
  misc_feature (106)..(108): Xaa can be any naturally occurring amino acid
  misc_feature (111)..(111): Xaa can be any naturally occurring amino acid
  misc_feature (113)..(113): Xaa can be any naturally occurring amino acid
  misc_feature (117)..(118): Xaa can be any naturally occurring amino acid
  misc_feature (120)..(121): Xaa can be any naturally occurring amino acid
  misc_feature (123)..(124): Xaa can be any naturally occurring amino acid
  misc_feature (126)..(127): Xaa can be any naturally occurring amino acid
  misc_feature (130)..(131): Xaa can be any naturally occurring amino acid
  misc_feature (153)..(153): Xaa can be any naturally occurring amino acid
  misc_feature (156)..(158): Xaa can be any naturally occurring amino acid
  misc_feature (161)..(161): Xaa can be any naturally occurring amino acid
  misc_feature (164)..(164): Xaa can be any naturally occurring amino acid
  misc_feature (168)..(169): Xaa can be any naturally occurring amino acid
  misc_feature (174)..(174): Xaa can be any naturally occurring amino acid
  misc_feature (180)..(180): Xaa can be any naturally occurring amino acid
  misc_feature (191)..(191): Xaa can be any naturally occurring amino acid
  misc_feature (195)..(195): Xaa can be any naturally occurring amino acid
  misc_feature (199)..(199): Xaa can be any naturally occurring amino acid
  misc_feature (206)..(206): Xaa can be any naturally occurring amino acid
  misc_feature (215)..(215): Xaa can be any naturally occurring amino acid
  misc_feature (217)..(217): Xaa can be any naturally occurring amino acid
YEEKVRPCIDLIDXRALGVEVEQDLALPAIAVIGDQSSGKSSVLGALSGVALPRGSGIVTRCPLVXKXXLXXXEXXWXGKVSXXDXEXEXSXXXXVEXXXXXXXQXXXAGXGXGISXXLXXLXXLXXSSXXVPDLTLIDLPGITRVAVGNQPXDIXXXIKXLIXKYIXXQETIXLVVVPXNVDIATTEALXMAQXVDPXGDRTIGXLTKPDLVDXGX

SEQ ID NO: 3 - protein, 289 aa, Artificial
  Synthetic peptide
  misc_feature (2)..(5): Xaa can be any naturally occurring amino acid
  misc_feature (8)..(8): Xaa can be any naturally occurring amino acid
  misc_feature (12)..(14): Xaa can be any naturally occurring amino acid
  misc_feature (29)..(29): Xaa can be any naturally occurring amino acid
  misc_feature (31)..(32): Xaa can be any naturally occurring amino acid
  misc_feature (36)..(37): Xaa can be any naturally occurring amino acid
  misc_feature (39)..(39): Xaa can be any naturally occurring amino acid
  misc_feature (41)..(41): Xaa can be any naturally occurring amino acid
  misc_feature (43)..(44): Xaa can be any naturally occurring amino acid
  misc_feature (47)..(51): Xaa can be any naturally occurring amino acid
  misc_feature (53)..(54): Xaa can be any naturally occurring amino acid
  misc_feature (58)..(58): Xaa can be any naturally occurring amino acid
  misc_feature (61)..(61): Xaa can be any naturally occurring amino acid
  misc_feature (64)..(64): Xaa can be any naturally occurring amino acid
  misc_feature (70)..(70): Xaa can be any naturally occurring amino acid
  misc_feature (73)..(73): Xaa can be any naturally occurring amino acid
  misc_feature (76)..(77): Xaa can be any naturally occurring amino acid
  misc_feature (82)..(82): Xaa can be any naturally occurring amino acid
  misc_feature (88)..(88): Xaa can be any naturally occurring amino acid
  misc_feature (91)..(94): Xaa can be any naturally occurring amino acid
  misc_feature (96)..(99): Xaa can be any naturally occurring amino acid
  misc_feature (106)..(106): Xaa can be any naturally occurring amino acid
  misc_feature (111)..(118): Xaa can be any naturally occurring amino acid
  misc_feature (120)..(121): Xaa can be any naturally occurring amino acid
  misc_feature (124)..(125): Xaa can be any naturally occurring amino acid
  misc_feature (128)..(129): Xaa can be any naturally occurring amino acid
  misc_feature (131)..(137): Xaa can be any naturally occurring amino acid
  misc_feature (139)..(139): Xaa can be any naturally occurring amino acid
  misc_feature (141)..(145): Xaa can be any naturally occurring amino acid
  misc_feature (149)..(151): Xaa can be any naturally occurring amino acid
  misc_feature (153)..(153): Xaa can be any naturally occurring amino acid
  misc_feature (156)..(157): Xaa can be any naturally occurring amino acid
  misc_feature (159)..(162): Xaa can be any naturally occurring amino acid
  misc_feature (164)..(165): Xaa can be any naturally occurring amino acid
  misc_feature (167)..(179): Xaa can be any naturally occurring amino acid
  misc_feature (183)..(183): Xaa can be any naturally occurring amino acid
  misc_feature (194)..(194): Xaa can be any naturally occurring amino acid
  misc_feature (196)..(197): Xaa can be any naturally occurring amino acid
  misc_feature (200)..(200): Xaa can be any naturally occurring amino acid
  misc_feature (202)..(202): Xaa can be any naturally occurring amino acid
  misc_feature (204)..(208): Xaa can be any naturally occurring amino acid
  misc_feature (212)..(212): Xaa can be any naturally occurring amino acid
  misc_feature (214)..(215): Xaa can be any naturally occurring amino acid
  misc_feature (218)..(227): Xaa can be any naturally occurring amino acid
  misc_feature (229)..(235): Xaa can be any naturally occurring amino acid
  misc_feature (237)..(238): Xaa can be any naturally occurring amino acid
  misc_feature (240)..(240): Xaa can be any naturally occurring amino acid
  misc_feature (243)..(244): Xaa can be any naturally occurring amino acid
  misc_feature (246)..(246): Xaa can be any naturally occurring amino acid
  misc_feature (250)..(252): Xaa can be any naturally occurring amino acid
  misc_feature (254)..(256): Xaa can be any naturally occurring amino acid
  misc_feature (259)..(259): Xaa can be any naturally occurring amino acid
  misc_feature (261)..(261): Xaa can be any naturally occurring amino acid
  misc_feature (263)..(264): Xaa can be any naturally occurring amino acid
  misc_feature (273)..(274): Xaa can be any naturally occurring amino acid
  misc_feature (280)..(280): Xaa can be any naturally occurring amino acid
  misc_feature (283)..(285): Xaa can be any naturally occurring amino acid
  misc_feature (287)..(289): Xaa can be any naturally occurring amino acid
EXXXXDVXRNLXXXLKKGYMIVKCRGQQXQXXLSLXXAXQXEXXFFXXXXXFXXLLEXGRXATXPCLAEXLTXELXXHICKXLPLLEXQIXXXXQXXXXELQKYGXDIPEXXXXXXXXLXXKIXXFNXXIXXXXXXXEXVXXXXXRLFXXXRXEFXXWXXXXEXXFXXXXXXXXXXXXXFENXYRGRELPGFVXYXXFEXIXKXXXXXLGGXAXXMLXXXXXXXXXXFXXXXXXXFXXFXNLXXTXKSKXXXIXXXQEXEXEXXIRLHFQMEXXVYCQDXVYXXXLXXX

SEQ ID NO: 4 - protein, 68 aa, Artificial
  Synthetic peptide
  misc_feature (2)..(4): Xaa can be any naturally occurring amino acid
  misc_feature (7)..(7): Xaa can be any naturally occurring amino acid
  misc_feature (10)..(11): Xaa can be any naturally occurring amino acid
  misc_feature (13)..(20): Xaa can be any naturally occurring amino acid
  misc_feature (27)..(27): Xaa can be any naturally occurring amino acid
  misc_feature (29)..(29): Xaa can be any naturally occurring amino acid
  misc_feature (31)..(31): Xaa can be any naturally occurring amino acid
  misc_feature (33)..(33): Xaa can be any naturally occurring amino acid
  misc_feature (35)..(38): Xaa can be any naturally occurring amino acid
  misc_feature (40)..(40): Xaa can be any naturally occurring amino acid
  misc_feature (47)..(52): Xaa can be any naturally occurring amino acid
  misc_feature (54)..(54): Xaa can be any naturally occurring amino acid
  misc_feature (56)..(56): Xaa can be any naturally occurring amino acid
  misc_feature (58)..(59): Xaa can be any naturally occurring amino acid
  misc_feature (62)..(63): Xaa can be any naturally occurring amino acid
  misc_feature (65)..(65): Xaa can be any naturally occurring amino acid
EXXXHLXAYXXEXXXXXXXXIPLIIQXFXLXTXGXXXXKXMLQLLQXXXXXXWXLXEXXDTXXKXKFL

SEQ ID NO: 5 - DNA, 2451 bp, Homo sapiens
ctattattgtatgactgactgctgaattacaccccaaaacaaccacctatactccccaaggccctgccatatcatatgcatattagaggcaatgaagcaccaaaatctaaggccagggaaggactgcggtggacaagggaagtgcctagggcgttaaaacttaaggaggcattgcaccaactgggagtgagctgtgtgtgctgtgtttcctgggcagggcgcctcactgcccttccgagtcctgccctggctgaggttgtgcctctatctggttatcagttgcctgcagctctttctgattttgttcgtgaacaagggcccttagggaagcctcccgctgttggaaacagcagcagcatcacccagagtgaagtctctgttcacatgagtgccagctctgctcacacctaccctgcgttcccaaggcactgttcctgcttcagaaagcatcagcccagcctgctcacagtcccccaggatgtgctgttgcctgggttcttgactaggaagtaaaaggcccagaggggttgagtgtgtgtgtgtgtgtgtgtgtgtgtgtgtgtgtgttaaagtgaggtcattgctaattcctgggagaacttgtgttccccttggatgaaccccttggatgaagaaccccttggatggggttgcccagccctaggagtggatcctgcaaagcaaaatagatttcagcacagggagctggctggggggctcctccccctcttgtaggtgcagcagtattgaatttgattccatccaggtttcccctgaccccaggacctaaagctggggcaggttctgggccttgaggttggttgggggtgacccttcctaatcatggaagtctttagcgggggcttgtaagggactgtgagctgagctaaggagaatggaggtggggtgccaaccgctcccaaagggaaatgccatctcccactcacagccagttagccgagaatgggagctcccaaagggaggaccacccatgggtctgcttgactcagccctccccaacccctttcacctttgcagtaaaactctagccaaggaagacaaagagacctttggagaccaaaacagaacttttaattcgggccaacagcaggctcatgcccaaaatggctgccaaccccaacaaagaaagcagctagcttatatgtcgtttgagatgggaaaaacaaggtaggatacaggtttcagacaaagacagtaaattacttaacccgtgacaattctgaggaaactggcaattcagttatattgactagtcatcctccaagctggactagggttggaggctggggtcccgaggcaggtgataagcttttgagataagcttgcatctgcaacttgttacaatgctgggaggggctgcttaaaattttagcctatgtgttacttctaaatagcttatactaaatgttaactgttttcatgtgcgtccatgtgaagagaccaccaaacaggctttgtgtgagcaacatggctgtgtatttcacctgggtgcaggcgggctgagtccgaaaagagagtcagcgaagggagataggggtggggccgttttataggatttgggaaggtaatggaaaattacagtcaaagggggttgttctctggtgggcaggggtgaatctcacaaagtacattctcaagggtggggagaattacaaataaccttcttaagggtggggaagattacaaagtacattgatcagttagggtggggcaggaacaaatcacaatggtggaatgtcatcagttaaggctgtttttacttcttttgtggatcttcagttactttaggccatctggatgtatacctgcaagtcacaggggatgcgatggcctggcctgggatgcgatggcctggcctgacaactattacctatgttatgtttattattttaagctttattattactattttatttattttattttattttccttccacacacccgtttccaccctggagaggccagatgagccagactccagggaggcctagaagtgggcaaggggaaacgggaaaggaggaagatggtatgggtgtgcctggttaggggtgggagtgctggacggagttcgggacaagaggggctctgcagccattggcacacaatgcctgggagtccctgctggtgctgggatcatcccagtgagccctgggagggaactgaagacccccaattaccaatgcatctgttttcaaaaccgacggggggaaggacatgcctaggttcaaggatacgtgcaggcttggatgactccgggccattagggagcctccggagcaccttgatcctcagacgggcctgatgaaacgagcatctgattcagcaggcctgggttcgggcccgagaacctgcgtctcccgcgagttcccgcgaggcaagtgctgcaggtgcggggccaggagctaggtttcgtttctgcgcccggagc

SEQ ID NO: 6 - DNA, 549 bp, Homo sapiens
cgccctcagcacagggtctgtgagtttcatttcttcgcggcgcggggcggggctgggcgcggggtgaaagaggcgaagcgagagcggaggccgcactccagcactgcgcagggaccggtgagtgtcgcttctgggggcagcgcccagtaaccgcgctaggagcgcggagaagggcattgggagagcggcgttcgtggcggagactagcgctccggagcacgggcacgacgggggcaccttctcggctgctagtaactaacaataataataatcataatcatagcaagggcgctgatgggcgggctcggagcacgcctgattctggttcccaccaggctgcccaggctcctgatgacgcatcagaaacatccccctaacccgcggccttcctgcaggagaggttgggaaggggtgggggacggggctcgggggaggtctccgagggactctagtaagcggggaagggcgccgggaaagtttcagatccacggctgcgcgggccacgagcccacccgaacgccgaccactgctttccgtcgacttctattt

SEQ ID NO: 7 - DNA, 5376 bp, Artificial
  Synthetic
ggtaccgagctccaccgcggtggcggccgctctagaactagtggatccagggaggcctagaagtgggcaaggggaaacgggaaaggaggaagatggtatgggtgtgcctggttaggggtgggagtgctggacggagttcgggacaagaggggctctgcagccattggcacacaatgcctgggagtccctgctggtgctgggatcatcccagtgagccctgggagggaactgaagacccccaattaccaatgcatctgttttcaaaaccgacggggggaaggacatgcctaggttcaaggatacgtgcaggcttggatgactccgggccattagggagcctccggagcaccttgatcctcagacgggcctgatgaaacgagcatctgattcagcaggcctgggttcgggcccgagaacctgcgtctcccgcgagttcccgcgaggcaagtgctgcaggtgcggggccaggagctaggtttcgtttctgctcccggagccgccctcagcacagggtctgtgagtttcatttcttcgcggcgcggggcggggctgggcgcggggtgaaagaggcgaacgagagcggtacgggctcgagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 8 - DNA, 20 bp, Artificial
  Synthetic
gcaagtgcaggtgccagaac

SEQ ID NO: 9 - DNA, 21 bp, Artificial
  Synthetic
cgtcttccatggtggctttac

(19) United States
(12) Patent Application Publication
(10) Pub. No.: US 2007/0275401 A1
(43) Pub. Date: Nov. 29, 2007

(54) USE OF Mx GTPASES IN THE PROGNOSIS AND TREATMENT OF CANCER

(75) Inventors: J. Frederic Mushinski, Bethesda, MD (US); Jane B. Trepel, Bethesda, MD (US); Michel Andre Horisberger, Allschwil (CH); Phuongmai Nguyen, Herndon, VA (US); Chand Khanna, Columbia, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: GOVERNMENT OF THE UNITED STATES OF AMERICA, REPRESENTED BY THE SECRETARY, DEP, Rockville, MD (US)

(21) Appl. No.: 11/781,399

(22) Filed: Jul. 23, 2007

Related U.S. Application Data
(62) Division of application No. 10/492,396.
(60) Provisional application No. 60/329,740, filed on Oct. 18, 2001.

(51) Int. Cl. C12Q 1/68

(52) U.S. Cl. 435/6

(57) ABSTRACT

The invention provides a method of reducing cancer progression comprising administering a Mx polypeptide or Mx-encoding nucleic acid to a host, such that the growth rate of the cancer cells is reduced, the metastatic potential of the cancer cells is reduced, or both. The invention also provides a method of assessing the metastatic potential of a cancer comprising (a) obtaining a sample of the cancer, (b) determining the level of Mx, Mx-nucleic acid, or both in the sample, and (c) comparing the level of Mx, Mx-encoding nucleic acid, or both with a control. In another aspect, the invention provides a method of assessing the ability of an agent to modulate the level of expression of an Mx comprising obtaining a cell expressing a known level of an Mx; contacting the cell with an agent to be tested; and assaying the cell for expression of the Mx to assess the ability of the agent to modulate Mx expression; alternatively, the method includes contacting a cell comprising a stable nucleic acid comprising the MxA promoter or other MxA regulatory sequence operably linked to one or more reporter genes to identify molecules that operably target such MxA nucleic acid sequences.

### CROSS-REFERENCE TO RELATED PATENT APPLICATIONS

This patent application claims the benefit of U.S. Provisional Patent Application No. 60/329,740, filed Oct. 18, 2001, which is incorporated by reference.

### FIELD OF THE INVENTION

This invention pertains to the use of Mx GTPases in the prognosis and treatment of cancer.

### BACKGROUND OF THE INVENTION

The American Cancer Society estimates the lifetime risk that an individual will develop cancer is 1 in 2 for men and 1 in 3 for women. The development of cancer, while still not completely understood, can be enhanced as a result of a variety of risk factors. For example, exposure to environmental factors (e.g., tobacco smoke) might trigger modifications in certain genes, thereby initiating cancer development. Alternatively, these cancer-predisposing genetic modifications may not occur as a result of exposure to environmental factors. Indeed, certain mutations (e.g., deletions, substitutions, etc.) can be inherited from generation to generation, thereby imparting an individual with a genetic predisposition to develop cancer.

Recent increases in the survival rates for many cancers have been linked to improvements in the detection of cancer at a stage at which treatment can be effective. Indeed, it has been noted that one of the most effective means to survive cancer is to detect its presence as early as possible. According to the American Cancer Society, the relative survival rate for many cancers would increase by about 15% if individuals participated in regular cancer screenings. Therefore, it is becoming increasingly useful to develop novel diagnostic and treatment tools to detect and treat the cancer either before it develops or at the earliest stage of development possible.

The Mx proteins, which also are known as the myxovirus (influenza) resistance proteins, are a family of unique GTPases. Several Mx proteins are known. Human MxA (also known as inducible protein p78 homolog) and murine p78 (Mx1) are the best-characterized members of the Mx family (see, e.g., Aebi et al., Mol. Cell. Biol., 9(11), 5062-72 (1989)). Human MxA (which also is referred to as Mx1) is a 78 kDa protein of 662 amino acids encoded by the IFI-78 (interferon-inducible 78 kDa protein) gene, which is located on the long arm of chromosome 21 (q22.3). MxA is produced in large amounts in the cytoplasm of certain cells treated with type-1 interferons (IFN-α and IFN-β). In this respect, MxA production has been shown to provide anti-RNA virus effects typically associated with type-1 interferons (see, e.g., Landis et al., J. Virol., 72(2), 1516-22 (1998) and Horisberg, Am. J. Respir. Crit. Care Med., 152(4), S67-71 (1995)). However, recent research suggests that the relationship between MxA and RNA virus resistance is not universal, consistent, or readily predictable (see, e.g., Pavlovic et al., Ciba Found. Symp., 176, 233-47 (1993), Thimme et al., Virology, 211(1), 296-301 (1995), Frese et al., Transgenic Res., 9(6), 429-38 (2000), Frese et al., J. Gen. Virol., 82(4), 723-33 (2001), and Leifeld et al., J. Pathol., 194(4), 478-83 (2001)).

Type-1 interferons are known to exhibit anti-cancer effects (see, e.g. U.S. Pat. Nos. 4,846,782, 4,997,645, 5,256,410, 5,480,640, and 6,207,145 and International Patent Application WO 82/00588). In this respect, MxA levels, in combination with tumor necrosis factor levels (TNF), have been used to identify patients who were most likely to benefit from IFN therapy (Bezares et al., J. Interferon. Cytokine Res., 16(7), 501-505 (1996)). However, researchers have failed to identify any correlation between MxA expression and therapeutic outcome in cells (Imam et al., Anticancer Res., 15(5B), 2191-95 (1995)). Furthermore, the prior art teaches that IFN-induced MxA expression in cancer cells is not involved in the antiproliferative action of IFN (Jakschies et al., J. Invest. Dermatol., 95(6 Suppl), 283S-241S (1990)). Thus, the art provides no suggestion that Mx GTPases are directly useful in the direct treatment or diagnosis of cancer.

Despite the success of interferon-based cancer treatments and related diagnostic techniques, there remains a need for improved and alternative ways to diagnose, prognosticate, and treat cancer. The invention provides novel methods of using Mx polypeptides and nucleic acids to accomplish these goals. These and other advantages of the invention, as well as additional inventive features, will be apparent from the description of the invention provided herein.

### BRIEF SUMMARY OF THE INVENTION

The present invention relates to the use of Mx GTPases (or “Mxs”) and Mx-encoding nucleic acids in the reduction of cancer progression and diagnosis of cancer. With respect to reducing cancer progression (or providing cancer treatment), the invention provides a method of reducing cancer progression, which includes administering an Mx or a nucleic acid encoding an Mx to a population of cancer cells, such that the growth rate of the cancer cells is reduced, the metastatic potential of the cancer cells is reduced, or both. In another exemplary aspect, the invention provides a method of reducing tumor progression comprising increasing the level of an Mx in a population of cancer cells having nominal physiological levels of type-1 interferons and IFN-γ such that the growth rate of the cancer cells is reduced, the metastatic potential of the cancer cells is reduced, or both.

With respect to diagnostic techniques, the invention provides, for example, a method of assessing the metastatic potential of a cancer comprising obtaining a sample of the cancer, determining the amount of an endogenous Mx, related Mx-encoding nucleic acid, or both in the sample, and assessing the metastatic potential of the cancer by comparing the level of endogenous Mx, Mx-encoding nucleic acid, or both in the sample with a control. The invention also provides a method of assessing the ability of an agent to affect the level of expression of an Mx comprising obtaining a cell expressing a known level of an Mx, contacting the cell with an agent to be tested, and assaying the cell for expression of the Mx to assess the ability of the agent to affect the level of expression of the Mx. In another aspect, the invention provides a method of assessing the metastatic potential of a cancer in a host by obtaining a sample of the cancer and assessing the metastatic potential of the cancer by determining the level of expression of Mx having a reduced GTPase activity, reduced tubulin association, or both in the sample as compared with wild-type Mx expressed in a non-cancerous cell of the host. In further aspects, the invention provides diagnostic techniques for identifying molecules that induce or inhibit expression of Mx nucleic acids (e.g., small molecule compounds that upregulate the MxA promoter).

### DETAILED DESCRIPTION OF THE INVENTION

The present invention provides a method of reducing cancer progression (e.g., tumor progression) comprising administering an Mx or a nucleic acid encoding an Mx to a population of cancer cells, or increasing expression of an Mx in such a population of cells, such that the growth rate of the cancer cells is reduced, the metastatic potential of the cancer cells is reduced, or both.

A “cancer cell” is any cell that divides and reproduces abnormally with uncontrolled growth (e.g., by exceeding the “Hayflick limit” of normal cell growth (as described in, e.g., Hayflick, Exp. Cell Res., 37, 614 (1965)). “Cancer progression,” as used herein, refers to any event or combination of events that promote, or which are indicative of, the transition of a normal, non-neoplastic cell to a cancerous, neoplastic cell. Examples of such events include phenotypic cellular changes associated with the transformation of a normal, non-neoplastic cell to a recognized pre-neoplastic phenotype, and cellular phenotypic changes that indicate transformation of a pre-neoplastic cell to a neoplastic cell. Aspects of cancer progression (also referred to herein as “cancer progression stages”) include cell crisis, immortalization and/or normal apoptotic failure, proliferation of immortalized and/or pre-neoplastic cells, transformation (i.e., changes which allow the immortalized cell to exhibit anchorage-independent, serum-independent and/or growth-factor independent, or contact inhibition-independent growth, or that are associated with cancer-indicative shape changes, aneuploidy, and focus formation), proliferation of transformed cells, development of metastatic potential, migration and metastasis (e.g., the disassociation of the cell from a location and relocation to another site), new colony formation, tumor formation, tumor growth, neotumorogenesis (formation of new tumors at a location distinguishable and not in contact with the source of the transformed cell(s)), and any combinations thereof. The methods of the present invention can be used to reduce, treat, prevent, or otherwise ameliorate any suitable aspect of cancer progression. The methods of the invention are particularly useful in the reduction and/or amelioration of tumor growth and metastatic potential, as described further herein. Methods that reduce, prevent, or otherwise ameliorate such aspects of cancer progression are preferred. A particularly preferred aspect of the invention is the reduction of the metastatic potential of cancer cells.

The detection of cancer progression can be achieved by any suitable technique, several examples of which are known in the art. Examples of suitable techniques include PCR and RT-PCR (e.g., of cancer cell associated genes or “markers”), biopsy, electron microscopy, positron emission tomography (PET), computed tomography, immunoscintigraphy and other scintegraphic techniques, magnetic resonance imaging (MRI), karyotyping and other chromosomal analysis, immunoassay/immunocytochemical detection techniques (e.g., differential antibody recognition), histological and/or histopathologic assays (e.g., of cell membrane changes), cell kinetic studies and cell cycle analysis, ultrasound or other sonographic detection techniques, radiological detection techniques, flow cytometry, endoscopic visualization techniques, and physical examination techniques. Examples of these and other suitable techniques are described in, e.g., Rieber et al., Cancer Res., 36(10), 3568-73 (1976), Brinkley et al., Tex. Rep. Biol. Med., 37, 26-44 (1978), Baky et al., Anal. Quant. Cytol., 2(3), 175-85 (1980), Laurence et al., Cancer Metastasis Rev., 2(4), 351-74 (1983), Cooke et al., Gut, 25(7), 748-55 (1984), Kim et al, Yonsel Med. J., 26(2), 167-74 (1985), Glaves, Prog. Clin. Biol. Res., 212, 151-67 (1986), McCoy et al., Immunol. Ser., 53, 171-87 (1990), Jacobsson et al., Med. Oncol. Tumor. Pharmacother., 8(4), 253-60 (1991), Swierenga et al., IARC Sci. Publ., 165-93 (1992), Himle, Lymphology, 27(3), 111-3 (1994), Laferte et al., J. Cell Biochem., 57(1), 101-19 (1995), Machiels et al., Eur. J. Cell Biochem., 66(3), 282-92 (1995), Chaiwun et al., Pathology (Phila), 4(1), 155-68 (1996), Jacobson et al, Ann. Oncol., 6(Suppl. 3), S3-8 (1996), Meijer et al., Eur. J. Cancer, 31A(7-8), 1210-11 (1995), Greenman et al., J. Clin. Endocrinol. Metab., 81(4), 1628-33 (1996), Ogunbiyi et al., Ann. Surg. Oncol., 4(8), 613-20 (1997), Merritt et al., Arch. Otolaryngol. Head Neck Surg., 123(2), 149-52 (1997), Bobardieri et al., Q. J. Nucl. Med., 42(1), 54-65 (1998), Giordano et al., J. Cell Biochem, 70(1), 1-7 (1998), Siziopikou et al., Breast J., 5(4), 221-29 (1999), Rasper, Surgery, 126(5), 827-8 (1999), von Knebel et al., Cancer Metastasis Rev., 18(1), 43-64 (1999), Britton et al., Recent Results Cancer Res., 157, 3-11 (2000), Caraway et al., Cancer, 90(2), 126-32 (2000), Castillo et al., Am. J. Neuroadiol., 21(5), 948-53 (2000), Chin et al., Mayo Clin. Proc., 75(8), 796-801 (2000), Kau et al., J. Ortohinolaryngol. Relat. Spe., 62(4), 199-203 (2000), Krag, Cancer J. Sci. Am., 6 (Suppl. 2), S121-24 (2000), Pantel et al., Curr. Opin. Oncol., 12(1), 95-101 (2000), Cook et al., Q. J. Nucl. Med., 45(1), 47-52 (2001), Gambhir et al., Clin. Nucl. Med., 26(10), 883-4 (2001), MacManus et al., Int. J. Radiat. Oncol. Biol. Phys., 50(2), 287-93 (2001), Olilla et al., Cancer Control., 8(5), 407-14 (2001), Taback et al., Recent Results Cancer Res., 158, 78-92 (2001), and references cited therein. Related techniques are described in U.S. Pat. Nos. 6,294,343, 6,245,501, 6,242,186, 6,235,486, 6,232,086, 6,228,596, 6,200,765, 6,187,536, 6,080,584, 6,066,449, 6,027,905, 5,989,815, 5,939,258, 5,882,627, 5,829,437, 5,677,125, and 5,455,159 and International Patent Applications WO 01/69199, WO 01/64110, WO 01/60237, WO 01/53835, WO 01/48477, WO 01/04353, WO 98/12564, WO 97/32009, WO 97/09925, and WO 96/15456.

A reduction of cancer progression can be any detectable decrease in (1) the rate of normal cells transforming to neoplastic cells (or any aspect thereof), (2) the rate of proliferation of pre-neoplastic or neoplastic cells, (3) the number of cells exhibiting a pre-neoplastic and/or neoplastic phenotype, (4) the physical area of a cell media (e.g., a cell culture, tissue, or organ (e.g., an organ in a mammalian host)) comprising pre-neoplastic and/or neoplastic cells, (5) the probability that normal cells will transform to neoplastic cells, (6) the probability that cancer cells will progress to the next aspect of cancer progression (e.g., a reduction in metastatic potential), or (7) any combination thereof. Such changes can be detected using any of the above-described techniques or suitable counterparts thereof known in the art, which are applied at a suitable time prior to the administration of the Mx GTPase, Mx-encoding nucleic acid, and/or increasing expression of host-native Mx and a suitable time thereafter, such that if a reduction in cancer occurs from the administration of the Mx GTPase, administration of the Mx-encoding nucleic acid, or increase in native Mx expression, it is detected. Times and conditions for assaying whether a reduction in cancer potential has occurred will depend on several factors including the type of cancer, type and amount of Mx administered or expressed, and the cancer progression stage assayed for. The ordinarily skilled artisan will be able to make appropriate determinations of times and conditions for performing such assays applying techniques and principles known in the art and/or routine experimentation.

The methods of the invention can be used to reduce the cancer progression of any suitable type of cancer. Advantageously, the methods of the invention can be used to reduce the cancer progression in prostate cancer cells, melanoma cells (e.g., cutaneous melanoma cells, ocular melanoma cells, and lymph node-associated melanoma cells), breast cancer cells, colon cancer cells, and lung cancer cells. The methods of the invention can be used to reduce cancer progression in both tumorigenic and non-tumorigenic cancers (e.g., non-tumor-forming hematopoietic cancers). For example, the methods of the invention can be applied to reduce the cancer progression of leukemia cells (e.g., acute lymphocytic leukemia, acute myeloid leukemia, chronic lymphocytic leukemia, and chronic myeloid leukemia). As discussed further herein, many of the therapeutic methods of the invention are applicable (and useful) in vitro, ex vivo, and/or in vivo. Thus, the invention in this respect provides method of administering a dose of an Mx GTPase (or other Mx peptide fragment), Mx-encoding nucleic acid, or combination thereof to a suitable cancer cell in culture (e.g., a HeLa cell, MCF-7 cell, HT 29 cell, Caco-2 cell, A549 cell, H460 cell, or Calu-1 cell), which can be used as a model for determining the effectiveness of the Mx and/or Mx-encoding nucleic acid (or particular dosage thereof) against a cancer cell type. Examples of suitable cancer cells are described in the ATCC catalog, an electronic copy of which is available at http://www.atcc.org/pdf/tcl.pdf. Further novel techniques relating to performing methods of the invention in vitro and/or ex vivo are discussed further herein.

In a first particular exemplary aspect, the invention provides a method of reducing cancer progression by administering an Mx GTPase (Mx) or a nucleic acid encoding an Mx to a population of cancer cells. An “Mx GTPase” is a protein comprising an amino acid sequence of at least about 300, desirably at least about 400, preferably at least about 500, and more preferably at least about 550 (e.g., about 550-700) amino acid residues that exhibits at least about 50%, desirably at least about 65%, preferably at least about 75%, more preferably at least about 90%, and even more preferably at least about 95% local or (preferably) overall (i.e., total) amino acid sequence identity to human MxA (as described in, e.g., Aebi et al., Mol. Cell. Biol., 9(11), 5062-72 (1989)). In the context of the present invention, an Mx GTPase can be any protein having the above-described structural features (e.g., at least about 80%, about 90-100%, or about 95-100% identity to MxA), which reduces cancer progression upon administration or expression of an effective amount of the Mx at, in, or near the cancer cells.

The Mx GTPase (which also may be referred to as the Mx protein) used in the methods of the invention typically and preferably is a naturally occurring (i.e., wild-type) Mx protein. Preferably, the Mx protein is a wild-type mammalian Mx protein. Advantageously, the Mx protein is a human MxA or a wild-type mammalian Mx that exhibits at least about 90% overall amino acid sequence homology (and, more preferably, at least about 90% amino acid sequence identity) to a human MxA. Human MxA includes any naturally expressed variants of MxA (e.g., MxAs expressed from either allele are suitable and naturally expressed truncated variants may be suitable). Human nucleic acid and amino acid sequences for MxA and related molecules are described under GenBank Accession Nos. NM_{—}0024642, M33882, AAA36458, NP_{—}002453, AAD43063, CAB90556, XP_{—}009773, A33481, AAA36337, and P20591. Examples of wild-type non-human MxA homologs are described in, e.g., Chesters et al., DNA Seq., 7(3-4), 239-42 (1997), Muller et al., J. Interferon Res., 12(2), 119-29 (1992), Jensen et al., J. Interferon. Cytokine Res., 20(8), 701-10 (2000), and Ellinwood et al., J. Interferon. Cytokine Res., 18(9), 745-55 (1998) and examples of nucleotide and amino acid sequences corresponding to such non-human wild-type homologs are described under GenBank Accession Nos. AAC23906, AAA31090, 146611, AAF44684, S21552, CAA46888, CAA36936, St 1736, NP_{—}058724, P79135, AF239823, X66093, AF047692, AF399856, NM_{—}013606, AB029920, U55216, M65087, BC007127, U88329, NW_{—}000110, and NM_{—}017028.

As mentioned above, the Mx protein can be any suitable synthetic MxA homolog. A synthetic MxA homolog preferably exhibits intrinsic GTPase activity similar to human MxA, and performs multiple rounds of GTP hydrolysis in the absence of accessory factors under conditions amenable to such GTPase activity. Thus, for example, the synthetic MxA homolog will exhibit a GTP/GDP affinity profile and conversion rate (as measured by, e.g., Kd and/or Km values) of within about 20% of the GTP/GDP affinity and conversion rate values of MxA (such values are described in, e.g., Horisberger, J. Viol., 66(8), 4705-9 (1992) and Richter et al., J. Biol. Chem, 270(22), 13512-17 (1995)). Desirably, the synthetic MxA homolog will have a mass of about 60-90 kDa, and more preferably about 70-80 kDa.

Preferably, the synthetic MxA homolog will form heteromultimers and/or homomultimers (with other Mx proteins) in vivo (MxA multimerization is described in, e.g., Paolo et al., J. Biol. Chem., 274(45), 32071-78 (1999), and references cited therein). Multimer formation can be determined by any suitable technique. Several suitable approaches to determining multimer formation are known in the art. A simple technique for assessing multimerization comprises subjecting a first portion of a composition comprising the putative multimer to size-exclusion chromatography, under conditions where the multimer will not be denatured, to determine the weight of the multimer. Another portion of the composition can be subjected to denaturing SDS-PAGE. If a multimer is formed the weights indicated in the two assays will be different, as the SDS-PAGE gel will exhibit a bond reflecting the weight of the monomeric fusion protein, rather than a multimer. Alternatively, two Western blots, one performed under denaturing conditions and the other under non-denaturing conditions can be performed on the multimer containing composition, if an antibody exhibits binding for both the multimer and the monomer. Recently, fluorescent microscopy, mass spectrometry, and light scattering techniques also have been used to determine multimerization. Alternatively, multimer-specific antibody binding assays can be used to assess multimerization. Other techniques related to determining multimer formation are described in, e.g., DiSalvo et al., Biol. Chem., 270, 7717-23 (1995), Cao et al., J. Biol. Chem., 271, 3154-62 (1996), and Olofsson et al., Proc. Natl. Acad. Sci. USA, 93, 2567-81 (1996)).

The synthetic MxA homolog will desirably comprise a dynamin GTPase domain (i.e., a domain that exhibits at least about 80% amino acid sequence homology and/or at least about 70% amino acid sequence identity (preferably about 90-100% identify) to the MxA dynamin GTPase domain (amino acids 46-257)), a dynamin central region domain (i.e., a domain that exhibits at least about 70% amino acid sequence homology and/or at least about 60% amino acid sequence identity to the MxA dynamin central region domain (amino acids 260-545 of MxA)), and/or a dynamin GTPase effector domain (i.e., a domain that exhibits at least about 80% amino acid sequence homology and/or at least about 70% amino acid sequence identity (preferably about 90-100% identity) to the MxA dynamin GTPase effector domain (amino acids 571-645 of MxA)). A MxA synthetic homolog or MxA fragment used in a therapeutic or diagnostic method of the invention desirably also or alternatively includes a domain having at least about 80%, at least about 85%, at least about 90%, at least about 95%, or more amino acid sequence identity to the carboxy-terminal domains responsible for oligomerization (see, e.g., Pontent et al., J. Virol. 71:2591-2599 (1997)). MxA homologs, variants, and/or fragments that contain sequences corresponding to the majority of the expressed MxA sequence (i.e., that exhibit a high level of total identify to MxA) are preferred.

A dynamin GTPase domain (or “GTPase domain”) preferably comprises a first GTP-binding region having a sequence in the pattern Gly Xaa Xaa Xaa Xaa Gly Lys Ser (SEQ ID NO: 1), a second GTP-binding region (positioned C-terminal to the first GTP-binding region) having a sequence in the pattern Asp Xaa Xaa Xaa Gly, and a third GTP-binding region (positioned C-terminal to the second GTP-binding region) having a sequence in the pattern Thr Lys Xaa Asp (Xaa throughout represents any amino acid, unless otherwise noted). The first GTP-binding region, and, more particularly, the Lys residue thereof, typically interacts with the beta and gamma phosphates of GTP.

More particularly, the dynamin GTPase domain preferably comprises a sequence within the sequence pattern Tyr Glu Glu Lys Val Arg Pro Cys Ile Asp Leu Ile Asp Xaa Arg Ala Leu Gly Val Glu Val Glu Gln Asp Leu Ala Leu Pro Ala Ile Ala Val Ile Gly Asp Gln Ser Ser Gly Lys Ser Ser Val Leu Gly Ala Leu Ser Gly Val Ala Leu Pro Arg Gly Ser Gly Ile Val Thr Arg Cys Pro Leu Val Xaa Lys Xaa Xaa Leu Xaa Xaa Xaa Glu Xaa Xaa Trp Xaa Gly Lys Val Ser Xaa Xaa Asp Xaa Glu Xaa Glu Xaa Ser Xaa Xaa Xaa Xaa Val Glu Xaa Xaa Xaa Xaa Xaa Xaa Xaa Gln Xaa Xaa Xaa Ala Gly Xaa Gly Xaa Gly Ile Ser Xaa Xaa Leu Xaa Xaa Leu Xaa Xaa Leu Xaa Xaa Ser Ser Xaa Xaa Val Pro Asp Leu Thr Leu Ile Asp Leu Pro Gly Ile Thr Arg Val Ala Val Gly Asn Gln Pro Xaa Asp Ile Xaa Xaa Xaa Ile Lys Xaa Leu Ile Xaa Lys Tyr Ile Xaa Xaa Gln Glu Thr Ile Xaa Leu Val Val Val Pro Xaa Asn Val Asp Ile Ala Thr Thr Glu Ala Leu Xaa Met Ala Gln Xaa Val Asp Pro Xaa Gly Asp Arg Thr Ile Gly Xaa Leu Thr Lys Pro Asp Leu Val Asp Xaa Gly Xaa (SEQ ID NO: 2), wherein Xaa can be any amino acid residue. The Mx alternatively or additionally desirably comprises a dynamin central region that comprises a sequence within the pattern Glu Xaa Xaa Xaa Xaa Asp Val Xaa Arg Asn Leu Xaa Xaa Xaa Leu Lys Lys Gly Tyr Met Ile Val Lys Cys Arg Gly Gln Gln Xaa Gln Xaa Xaa Leu Ser Leu Xaa Xaa Ala Xaa Gln Xaa Glu Xaa Xaa Phen Phe Xaa Xaa Xaa Xaa Xaa Phen Xaa Xaa Leu Leu Glu Xaa Gly Arg Xaa Ala Thr Xaa Pro Cys Leu Ala Glu Xaa Leu Thr Xaa Glu Leu Xaa Xaa H is Ile Cys Lys Xaa Leu Pro Leu Leu Glu Xaa Gln Ile Xaa Xaa Xaa Xaa Gln Xaa Xaa Xaa Xaa Glu Leu Gln Lys Tyr Gly Xaa Asp Ile Pro Glu Xaa Xaa Xaa Xaa Xaa Xaa Xaa Xaa Leu Xaa Xaa Lys Ile Xaa Xaa Phen Asn Xaa Xaa Ile Xaa Xaa Xaa Xaa Xaa Xaa Xaa Glu Xaa Val Xaa Xaa Xaa Xaa Xaa Arg Leu Phe Xaa Xaa Xaa Arg Xaa Glu Phe Xaa Xaa Trp Xaa Xaa Xaa Xaa Glu Xaa Xaa Phen Xaa Xaa Xaa Xaa Xaa Xaa Xaa Xaa Xaa Xaa Xaa Xaa Xaa Phe Glu Asn Xaa Tyr Arg Gly Arg Glu Leu Pro Gly Phe Val Xaa Tyr Xaa Xaa Phen Glu Xaa Ile Xaa Lys Xaa Xaa Xaa Xaa Xaa Leu Gly Gly Xaa Ala Xaa Xaa Met Leu Xaa Xaa Xaa Xaa Xaa Xaa Xaa Xaa Xaa Xaa Phe Xaa Xaa Xaa Xaa Xaa Xaa Xaa Phe Xaa Xaa Phe Xaa Asn Leu Xaa Xaa Thr Xaa Lys Ser Lys Xaa Xaa Xaa Ile Xaa Xaa Xaa Gln Glu Xaa Glu Xaa Glu Xaa Xaa Ile Arg Leu H is Phe Gln Met Glu Xaa Xaa Val Tyr Cys Gln Asp Xaa Val Tyr Xaa Xaa Xaa Leu Xaa Xaa Xaa (SEQ ID NO: 3). The Mx further additionally or alternatively comprises a GTPase effector domain that has a sequence within the sequence pattern Glu Xaa Xaa Xaa H is Leu Xaa Ala Tyr Xaa Xaa Glu Xaa Xaa Xaa Xaa Xaa Xaa Xaa Xaa Ile Pro Leu Ile Ile Gln Xaa Phe Xaa Leu Xaa Thr Xaa Gly Xaa Xaa Xaa Xaa Lys Xaa Met Leu Gln Leu Leu Gln Xaa Xaa Xaa Xaa Xaa Xaa Trp Xaa Leu Xaa Glu Xaa Xaa Asp Thr Xaa Xaa Lys Xaa Lys Phe Leu (SEQ ID NO: 4).

The MxA synthetic homolog desirably has also or alternatively has a C-terminal half which comprises a LZ1 domain (i.e., an amino acid sequence that exhibits at least about 90% homology and/or at least about 80% identity (preferably at least about 90% identity) to the MxA LZ1 domain (amino acids 362-415 of MxA)), as well as a sequence that exhibits at least about 90% homology and/or at least about 80% identity to amino acids 363-415 of MxA. More generally, the C-terminus half of the MxA synthetic homolog will preferably comprise an amino acid sequence of at least about 200 amino acid residues that exhibits at least about 80% homology and/or at least about 70% identity (preferably at least about 90% identity) to amino acids 362-574 of MxA, which promotes intermolecular interaction in the protein and formation of multimers (related sequences and their functions are described in, e.g., Schwemmle et al., J. Biol. Chem., 270(22), 13518-23 (1995) and Paolo et al., J. Biol. Chem., 274(45), 32071-78 (1999).

The Mx protein of the invention is preferably a functional GTPase. GTPase activity of an Mx protein can be assessed by any suitable technique. Desirably, the Mx protein exhibits at least about 65% (preferably at least about 75%, more preferably at least about 90%, or even at least about 95%) of the GTPase activity of human MxA (based on, e.g., GDP-GTP conversions per minute). Methods of assaying GTPase activity are described in Ferguson et al., J. Biol. Chem., 261, 7393-99 (1986) and U.S. Pat. No. 5,589,568. The GTPase activity of naturally occurring Mx proteins is described in several of the references cited herein.

“Identity” (sometimes referred to as “overall” identity) as used herein with respect to amino acid or polynucleotide sequences refers to the percentage of residues or bases that are identical in the two sequences when the sequences are optimally aligned. If, in the optimal alignment, a position in a first sequence is occupied by the same amino acid residue or nucleotide as the corresponding position in the second sequence, the sequences exhibit identity with respect to that position. The level of identity between two sequences (or “percent sequence identity”) is measured as a ratio of the number of identical positions shared by the sequences with respect to the size of the sequences (i.e., percent sequence identity=(number of identical positions/total number of positions)×100).

The “optimal alignment” is the alignment that provides the highest identity between the aligned sequences. In obtaining the optimal alignment, gaps can be introduced, and some amount of non-identical sequences and/or ambiguous sequences can be ignored. Preferably, if a gap needs to be inserted into a first sequence to achieve the optimal alignment, the percent identity is calculated using only the residues that are paired with a corresponding amino acid residue (i.e., the calculation does not consider residues in the second sequences that are in the “gap” of the first sequence). However, it is often preferable that the introduction of gaps and/or the ignoring of non-homologous/ambiguous sequences are associated with a “gap penalty.”

A number of mathematical algorithms for rapidly obtaining the optimal alignment and calculating identity between two or more sequences are known and incorporated into a number of available software programs. Examples of such programs include the MATCH-BOX, MULTAIN, GCG, FASTA, and ROBUST programs for amino acid sequence analysis, and the SIM, GAP, NAP, LAP2, GAP2, and PIPMAKER programs for nucleotide sequences. Preferred software analysis programs for both amino acid and polynucleotide sequence analysis include the ALIGN, CLUSTAL W (e.g., version 1.6 and later versions thereof), and BLAST programs (e.g., BLAST 2.1, BL2SEQ, and later versions thereof).

For amino acid sequence analysis, a weight matrix, such as the BLOSUM matrixes (e.g., the BLOSUM45, BLOSUM50, BLOSUM62, and BLOSUM80 matrixes), Gonnet matrixes (e.g., the Gonnet40, Gonnet80, Gonnet120, Gonnet160, Gonnet250, and Gonnet350 matrixes), or PAM matrixes (e.g., the PAM30, PAM70, PAM120, PAM160, PAM250, and PAM350 matrixes), are used in determining identity. BLOSUM matrixes are preferred. The BLOSUM50 and BLOSUM62 matrixes are typically most preferred. In the absence of availability of such weight matrixes (e.g., in nucleic acid sequence analysis and with some amino acid analysis programs), a scoring pattern for residue/nucleotide matches and mismatches can be used (e.g., a +5 for a match and −4 for a mismatch pattern).

The ALIGN program produces an optimal global alignment of the two chosen protein or nucleic acid sequences using a modification of the dynamic programming algorithm described by Myers and Miller, CABIOS, 4, 11-17 (1988). Preferably, if available, the ALIGN program is used with weighted end-gaps. If gap opening and gap extension penalties are available, they are preferably set between about −5 to −15 and 0 to −3, respectively, more preferably about −12 and −0.5 to −2, respectively, for amino acid sequence alignments, and −10 to −20 and −3 to −5, respectively, more preferably about −16 and −4, respectively, for nucleic acid sequence alignments. The ALIGN program and principles underlying it are further described in, e.g., Pearson et al., Proc. Natl. Acad. Sci. USA, 85, 2444-48 (1988), and Pearson et al., Methods Enzymol., 183, 63-98 (1990).

The BLAST programs provide analysis of at least two amino acid or nucleotide sequences, either by aligning a selected sequence against multiple sequences in a database (e.g., GenSeq), or, with BL2SEQ, between two selected sequences. BLAST programs are preferably modified by low complexity filtering programs such as the DUST or SEG programs, which are preferably integrated into the BLAST program operations (see, e.g., Wooton et al., Compu. Chem., 17, 149-63 (1993), Altschul et al., Nat. Genet., 6, 119-29 (1994), Hancock et al., Comput. Appl. Biosci., 10, 67-70 (1994), and Wootton et al., Meth. in Enzym., 266, 554-71 (1996)). If a lambda ratio is used, preferred settings for the ratio are between 0.75 and 0.95, more preferably between 0.8 and 0.9. If gap existence costs (or gap scores) are used, the gap existence cost preferably is set between about −5 and −15, more preferably about −10, and the per residue gap cost preferably is set between about 0 to −5, more preferably between 0 and −3 (e.g., −0.5). Similar gap parameters can be used with other programs as appropriate. The BLAST programs and principles underlying them are further described in, e.g., Altschul et al., J. Mol. Biol., 215, 403-10 (1990), Karlin and Altschul, Proc. Natl. Acad. Sci. USA, 87, 2264-68 (1990) (as modified by Karlin and Altschul, Proc. Natl. Acad. Sci. USA, 90, 5873-77 (1993)), and Altschul et al., Nucl. Acids Res., 25, 3389-3402 (1997)).

For multiple sequence analysis, the CLUSTAL W program can be used. The CLUSTAL W program desirably is run using “dynamic” (versus “fast”) settings. Preferably, nucleotide sequences are compared using the BESTFIT matrix, whereas amino acid sequences are evaluated using a variable set of BLOSUM matrixes depending on the level of identity between the sequences (e.g., as used by the CLUSTAL W version 1.6 program available through the San Diego Supercomputer Center (SDSC)). Preferably, the CLUSTAL W settings are set to the SDSC CLUSTAL W default settings (e.g., with respect to special hydrophilic gap penalties in amino acid sequence analysis). The CLUSTAL W program and underlying principles of operation are further described in, e.g., Higgins et al., CABIOS, 8(2), 189-91 (1992), Thompson et al., Nucleic Acids Res., 22, 4673-80 (1994), and Jeanmougin et al., Trends Biochem. Sci., 23, 403-07 (1998).

“Local sequence identity” refers to identity between portions of two amino acid or nucleic acid sequences. Local sequence identity can be determined using local sequence alignment software, e.g., the BLAST programs described above, the LFASTA program, or, more preferably, the LALIGN program. Preferably, the LALIGN program using a BLOSUM50 matrix analysis is used for amino acid sequence analysis, and a +5 match/−4 mismatch analysis is used for polynucleotide sequence analysis. Gap extension and opening penalties are preferably the same as those described above with respect to analysis with the ALIGN program. For LALIGN (or other program) analysis using k-tup value settings (also referred to as “k-tuple” or ktup values), a k-tup value of 0-3 for proteins, and 0-10 (e.g., about 6) for nucleotide sequences, is preferred.

Several commercially available software suites incorporate the ALIGN, BLAST, and CLUSTAL W programs and similar functions, and may include significant improvements in settings and analysis. Examples of such programs include the GCG suite of programs and those available through DNASTAR, Inc. (Madison, Wis.). Particular preferred programs include the Lasergene and Protean programs sold by DNASTAR.

Because various algorithms, matrixes, and programs are commonly used to analyze sequences, amino acid and polynucleotide sequences are preferably characterized in terms of approximate identities by indicating a range of identity “about” a particular identity (e.g., +/−10%, more preferably +/−8%, and even more preferably +/−5% of the particular identity). Alternatively, an exact identity can be measured by using only one of the aforementioned programs, preferably one of the BLAST programs, as described herein.

Amino acid sequence “homology,” as used herein, is a function of the number of corresponding conserved and identical amino acid residues in the optimal homology alignment. The “optimal homology alignment” is the alignment that provides the highest level of homology (i.e., functional residue homology) between two amino acid sequences, using the principles described above with respect to the “optimal alignment.” Conservative amino acid residue substitutions involve exchanging a member within one class of amino acid residues for a residue that belongs to the same class. MxA synthetic homologs having sequence containing a high percentage of conservative substitutions are expected to substantially retain the biological properties and functions associated with their wild-type counterpart or wild-type counterpart portions. The classes of amino acids and the members of those classes are presented in Table 1.

TABLE 1
Amino Acid Residue Classes

| Amino Acid Class | Amino Acid Residues |
|---|---|
| Acidic Residues | ASP and GLU |
| Basic Residues | LYS, ARG, and HIS |
| Hydrophilic Uncharged Residues | SER, THR, ASN, and GLN |
| Aliphatic Uncharged Residues | GLY, ALA, VAL, LEU, and ILE |
| Non-polar Uncharged Residues | CYS, MET, and PRO |
| Aromatic Residues | PHE, TYR, and TRP |

An MxA synthetic homolog also desirably exhibits high weight homology to human MxA. “High weight homology” means that at least about 40%, preferably at least about 60%, and more preferably at least about 70% (e.g., about 80%-95%) of the non-identical amino acid residues are members of the same weight-based “weak conservation group” or “strong conservation group” as the corresponding amino acid residue in human MxA (in the optimal alignment or an alignment optimal for weight group conservation). Strong group conservation is preferred. Weight-based conservation is determined on the basis of whether the non-identical corresponding amino acid is associated with a positive score on one of the weight-based matrices described herein (e.g., the BLOSUM50 matrix and preferably the PAM250 matrix). Weight-based strong conservation groups include Ser Thr Ala, Asn Glu Gln Lys, Asn H is Gln Lys, Asn Asp Glu Gln, Gln H is Arg Lys, Met Ile Leu Val, Met Ile Leu Phe, H is Tyr, and Phe Tyr Trp. Weight-based weak conservation groups include Cys Ser Ala, Ala Thr Val, Ser Ala Gly, Ser Thr Asn Lys, Ser Thr Pro Ala, Ser Gly Asn Asp, Ser Asn Asp Glu Gln Lys, Asn Asp Glu Gln H is Lys, Asn Glu Gln H is Arg Lys, Phe Val Leu Ile Met, and H is Phe Tyr. The CLUSTAL W sequence analysis program provides analysis of weight-based strong conservation and weak conservation groups in its output, and offers the preferred technique for determining weight-based conservation, preferably using the CLUSTAL W default settings used by SDSC.

Preferably, an MxA synthetic homolog comprises a hydropathy profile (hydrophilicity) similar to that of human MxA. A hydropathy profile can be determined using the Kyte & Doolittle index, the scores for each naturally occurring amino acid in the index being as follows: I (+4.5), V (+4.2), L (+3.8), F (+2.8), C (+2.5), M (+1.9); A (+1.8), G (−0.4), T (−0.7), S (−0.8), W (−0.9), Y (−1.3), P (−1.6), H (−3.2); E (−3.5), Q (−3.5), D (−3.5), N (−3.5), K (−3.9), and R (−4.5) (see, e.g., U.S. Pat. No. 4,554,101 and Kyte & Doolittle, J. Molec. Biol., 157, 105-32 (1982) for further discussion). Preferably, at least about 45%, preferably at least about 60%, and more preferably at least about 75% (e.g., at least about 85%, at least about 90%, or at least about 95%) of the amino acid residues which differ from the corresponding residues in MxA (in one of the aforementioned optimal alignments) exhibit less than a +/−2 change in hydrophilicity, more preferably less than a +/−1 change in hydrophilicity, and even more preferably less than a +/−0.5 change in hydrophilicity. Overall, the MxA synthetic homolog preferably exhibit a total change in hydrophilicity of less than about 150, more preferably less than about 100, and even more preferably less than about 50 (e.g., less than about 30, less than about 20, or less than about 10) with respect to human MxA. Examples of typical amino acid substitutions that retain similar or identical hydrophilicity include arginine-lysine substitutions, glutamate-aspartate substitutions, serine-threonine substitutions, glutamine-asparagine substitutions, and valine-leucine-isoleucine substitutions. The GREASE program, available through the SDSC, provides a convenient way for quickly assessing the hydropathy profile of a peptide portion.

MxA homologs (both synthetic and naturally occurring) can comprise or consist of a peptide of at least about 300 amino acid residues, preferably at least about 400 amino acid residues, and more preferably at least about 500 (e.g., at least about 550, at least about 600, or more) amino acid residues encoded by a polynucleotide that hybridizes to (1) the complement of a polynucleotide that, when expressed, produces a human MxA protein, under at least moderate, preferably high, stringency conditions, or (2) a polynucleotide which would hybridize to the complement of such a sequence under such conditions but for the degeneracy of the genetic code.

Exemplary moderate stringency conditions include overnight incubation at 37° C. in a solution comprising 20% formamide, 0.5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C., or substantially similar conditions, e.g., the moderately stringent conditions described in Sambrook et al., Molecular Cloning: A Laboratory Manual (Cold Spring Harbor Press 1989). High stringency conditions are conditions that use, for example, (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride, 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS, and 10% dextran sulfate at 42° C., with washes at (i) 42° C. in 0.2×SSC, (ii) at 55° C. in 50% form amide and (iii) at 55° C. in 0.1×SSC preferably in combination with EDTA). Additional details and explanation of stringency of hybridization reactions are provided in, e.g., Ausubel et al., Current Protocols in Molecular Biology (Wiley Interscience Publishers 1995).

Desirably, an MxA synthetic homolog will comprise at least one amino acid sequence that is bound by an antibody that also binds a wild-type Mx protein, and, more preferably, an antibody that binds human MxA. Methods for obtaining antibodies that can be applied to Mx proteins are known in the art (see, e.g., Gavilodono et al., Biotechniques, 29(1), 128-32, 134-6, and 138 (passim) (2000), Nelson et al., Mol. Pathol., 53(3), 111-7 (2000), Laurino et al., Ann. Clin. Lab. Sci., 29(3), 158-66 (1999), Rapley, Mol. Biotechnol., 3(2), 139-54 (1995), Zaccolo et al., Int. J. Clin. Lab. Res., 23(4), 192-8 (1993), Morrison, Annu. Rev. Immunol., 10, 239-65 (1992), “Antibodies, Annigene, and Molecular Mimiery,” Meth. Enzymd., 178 (John J. Langone, Ed. 1989), Moore, Clin. Chem., 35(9), 1849-53 (1989), Rosalki et al., Clin. Chim. Acta, 183(1), 45-58 (1989), and Tami et al., Am. J. Hosp. Pharm., 43(11), 2816-25 (1986), as well as U.S. Pat. Nos. 4,022,878, and 4,350,683). A preferred technique for producing antibodies is provided in Border et al., Proc. Natl. Acad. Sci., USA, 97(20), 10701-05 (2000). Antibodies specific to Mx proteins are described in, e.g., Towbin et al., J. Interferon Res., 12(2), 67-74 (1992) and Flohr, FEBS Lett., 463(1-2), 24-8 (1999), as well as U.S. Pat. Nos. 6,180,102 and 6,200,559.

A MxA synthetic homolog will desirably comprise a peptide portion (amino acid sequence or polypeptide subunit) or, more typically, be a polypeptide that exhibits structural homology (or “structural similarity”) to a wild-type Mx protein, preferably to human MxA. Structural homology can be determined by any suitable technique, preferably using a suitable software program for making such assessments. Examples of such programs include the MAPS program and the TOP program (described in Lu, Protein Data Bank Quarterly Newsletter, #78, 10-11(1996), and Lu, J. Appl. Cryst., 33, 176-183(2000)). The MxA synthetic homolog will desirably exhibit low topological diversity (e.g., a topical diversity of less than about 20, preferably less than about 15, and more preferably less than about 10), or both, with respect to human MxA. Alternatively, structural similarity can be assessed by comparing the amino acid sequence of the synthetic MxA homolog to human MxA using the PROCHECK program (described in, e.g., Laskowski, J Appl. Cryst, 26, 283-291 (1993)), the MODELLER program, or commercially available programs incorporating such features. Alternatively, a sequence comparison using a program such as the PredictProtein server (available at http://dodo.cpmc.columbia.edu/predictprotein/) can identify the level of structural similarity between the synthetic MxA homolog and human MxA. Additional techniques for analyzing protein structure that can be applied to determine whether the MxA homolog exhibits a suitable level of structural similarity to a wild-type Mx protein such as human MxA are described in, e.g., Yang and Honig, J. Mol. Biol., 301(3), 665-78 (2000), Aronson et al., Protein Sci., 3(10), 1706-11 (1994), Marti-Remon et al., Annu. Rev. Biophys. Biomol. Struct., 29, 291-325 (2000), Halaby et al., Protein Eng., 12(7), 563-71 (1999), Basham, Science, 283, 1132 (1999), Johnston et al., Crit. Rev. Biochem. Mol. Biol., 29(1), 1-68 (1994), Moult, Curr. Opin. Biotechnol., 10(6), 583-6 (1999), Benner et al., Science, 274, 1448-49 (1996), and Benner et al., Science, 273, 426-8 (1996).

MxA synthetic homologs desirably associate with the cytoskeleton, and, in particular, tubulin, at levels similar to wild-type MxA (e.g., by exhibiting an association of at least about 80%, preferably at least about 90% of the affinity of human MxA exhibits for tubulin). Measuring protein affinity is well known in the art, and specific techniques related to MxA and tubulin association are described elsewhere herein.

As an alternative, or in addition to, the above-described Mx protein delivery/administration techniques (examples of which are described elsewhere herein), the method can include delivering a polynueleotide encoding the Mx protein to the cancer cells. The polynucleotide sequence can be any suitable nucleotide sequence (e.g., single stranded or double stranded RNA, DNA, or combinations thereof) and can include any suitable nucleotide base, base analog, and/or backbone (e.g., a backbone formed by, or including, a phosphothioate, rather than phosphodiester, linkage). Examples of suitable modified nucleotides which can be incorporated in the polynucleotide sequence are provided in the Manual of Patent Examining Procedure § 2422 (7th Revision—2000). The polynucleotide sequence can be any suitable length, but preferably is at least about 1200 nucleotides (nt) in length, more preferably at least about 1500 nt, and even more preferably at least about 1800 nt. The polynucleotide sequence can comprise any sequence of nucleic acids that results in the production of the Mx protein. As such, the polynucleotide sequence is not limited to sequences that directly code for production of the Mx protein. For example, the polynucleotide can comprise a sequence that contains self-splicing introns (or other self-spliced RNA transcripts) that form the peptide portions and/or a fusion protein (as described in, e.g., U.S. Pat. No. 6,010,884). The polynucleotides also can comprise sequences which result in other splice modifications at the RNA level to produce an mRNA transcript encoding a fusion protein and/or at the DNA level by way of trans-splicing mechanisms prior to transcription (as described in, e.g., Chabot, Trends Genet., 12(11), 472-78 (1996), Cooper, Am. J. Hum. Genet., 61(2), 259-66 (1997), and Hertel et al., Curr. Opin. Cell. Biol., 9(3), 350-57 (1997)).

The polynucleotide can comprise a codon optimized portion or codon optimized sequence. Codon optimization, as used herein, refers both to optimizing (through replacement) the polynucleotide sequence with respect to both to host (e.g., human) codon frequency and/or codon pair (i.e., codon context) optimized for a particular species, by using techniques such as those described in Buckingham et al., Biochimie, 76(5), 351-54 (1994) and U.S. Pat. Nos. 5,082,767, 5,786,464, and 6,114,148. Additionally, a codon optimized Mx-encoding polynucleotide sequence can be generated by subjecting the amino acid sequences of the desired Mx protein to backtranslation using a suitable program, such as the Entelechon backtranslation tool (available at http://www.entelechon.com/eng/backtranslation.html). Resulting nucleotide sequences can be produced through standard polynucleotide synthesis techniques. Partially codon optimized sequences also can be used, such as codon sequences where only some or all of the “rarest” sequences (for the particular organism of interest) are removed. For example, a human MxA-encoding sequence can be generated by modifying the human MxA gene sequence through the replacement of at least one (preferably all) of the Ala-encoding GCA and/or GCT codons with GCC codons.

Production of the Mx-encoding polynucleotide can be accomplished by any suitable technique. Recombinant polynucleotide production is well understood, and methods of producing such molecules are provided in, e.g., Mulligan, Science 260, 926-932 (1993), Friedman, Therapy For Genetic Diseases (Oxford University Press, 1991), Ibanez et al., EMBO J., 10, 2105-10 (1991), Ibanez et al., Cell, 69, 329-41 (1992), and U.S. Pat. Nos. 4,440,859, 4,530,901, 4,582,800, 4,677,063, 4,678,751, 4,704,362, 4,710,463, 4,757,006, 4,766,075, and 4,810,648, and are more particularly described in Sambrook and Ausubel, supra.

A number of MxA synthetic homolog-encoding sequences can be generated by way of mutagenesis, directed evolution, or related techniques. For example, homolog-encoding sequences can be obtained through application of site-directed mutagenesis (as described in, e.g., Edelman et al., DNA, 2, 183 (1983), Zoller et al., Nucl. Acids Res., 10, 6487-5400 (1982), and Veira et al., Meth. Enzymol., 153, 3 (1987)), alanine scanning, or random mutagenesis, such as iterated random point mutagenesis induced by error-prone PCR, chemical mutagen exposure applied to wild-type MX protein-encoding gene sequences, or through wild-type polynucleotide expression in mutator cells (see, e.g., Bornscheuer et al., Biotechnol. Bioeng., 58, 554-59 (1998), Cadwell and Joyce, PCR Methods Appl., 3(6), S136-40 (1994), Kunkel et al., Methods Enzymol., 204, 125-39 (1991), Low et al., J. Mol. Biol., 260, 359-68 (1996), Taguchi et al., Appl. Environ. Microbiol., 64(2), 492-95 (1998), and Zhao et al., Nat. Biotech., 16, 258-61 (1998)). Suitable primers for PCR-based site-directed mutagenesis or related techniques can be prepared by the methods described in, e.g., Crea et al., Proc. Natl. Acad. Sci. USA, 75, 5765 (1978).

Other polynucleotide mutagenesis methods useful for producing novel MxA synthetic homologs and related polynucleotides include PCR mutagenesis techniques (as described in, e.g., Kirsch et al., Nucl. Acids Res., 26(7), 1848-50 (1998), Seraphin et al., Nucl. Acids Res., 24(16), 3276-7 (1996), Caldwell et al., PCR Methods Appl., 2(1), 28-33 (1992), Rice et al., Proc. Natl. Acad. Sci. USA. 89(12), 5467-71 (1992) and U.S. Pat. No. 5,512,463), cassette mutagenesis techniques based on the methods described in Wells et al., Gene, 34, 315 (1985), phagemid display techniques (as described in, e.g., Soumiltion et al., Appl. Biochem. Biotechnol., 47, 175-89 (1994), O'Neil et al., Curr. Opin. Struct. Biol., 5(4), 443-49 (1995), Dunn, Curr. Opin. Biotechnol., 7(5), 547-53 (1996), and Koivunen et al., J. Nucl. Med., 40(5), 883-88 (1999)), reverse translation evolution (as described in, e.g., U.S. Pat. No. 6,194,550), saturation mutagenesis described in, e.g., U.S. Pat. No. 6,171,820), PCR-based synthesis shuffling (as described in, e.g., U.S. Pat. No. 5,965,408) and recursive ensemble mutagenesis (REM) (as described in, e.g., Arkin and Yourvan, Proc. Natl. Acad. Sci. USA, 89, 7811-15 (1992), and Delgrave et al., Protein Eng., 6(3), 327-331 (1993)). Alternatively, the MxA synthetic homolog can pre-designed and synthetically produced using techniques such as those described in, e.g., Itakura et al., Annu. Rev. Biochem., 53, 323 (1984), Itakura et al., Science, 198, 1056 (1984), and Ike et al., Nucl. Acid Res., 11, 477 (1983).

Alternatively, the MxA synthetic homolog-encoding polynucleotide can be obtained through application of directed evolution techniques to wild-type Mx protein-encoding sequences (e.g., synthetic polynucleotide shuffling). Examples of such techniques are described in, e.g., Stemmer, Nature, 370, 389-91 (1994), Cherry et al., Nat. Biotechnol. 17, 379-84 (1999), and Schmidt-Dannert et al., Nat Biotechnol, 18(7), 750-53 (2000). Preferably, shuffling is performed in combination with staggered extension (StEP), random primer shuffling, backcrossing of improved variants, or any combination thereof e.g., as described in Zhao et al., supra, Cherry et al., supra, Arnold et al., Biophys. J., 73, 1147-59 (1997), Zhao and Arnold, Nucl. Acids Res., 25(6), 1307-08 (1997), and Shao et al., Nucl. Acids Res., 26, 681-83 (1998). Alternatively, the incremental truncation for the creation of hybrid enzymes (ITCHY) method (see, e.g., Ostermeier et al., Nat. Biotechnol., 17(12), 1205-09 (1999)) can be applied to produce novel MxA synthetic homologs.

An Mx-encoding polynucleotide typically includes or is functionally associated with one or more suitable “expression control sequences” operably linked to the sequence encoding the Mx protein. An expression control sequence is any nucleotide sequence that assists or modifies the expression (e.g., the transcription, translation, or both) of the nucleic acid encoding the Mx protein. The expression control sequence can be naturally associated with a polynucleotide encoding a wild-type Mx (e.g., a human MxA promoter (as described in, e.g., Chang et al., Arch Virol., 117(1-2), 1-15 (1991) and Nakade et al., FEBS Lett., 418(3), 315-8 (1997), and recorded under GenBank Accession No. X55639). Alternatively or additionally, the polynucleotide can comprise any suitable number of heterologous expression control sequences (e.g., a synthetic variant of an MxA promoter sequence). For example, the Mx-encoding sequence of the polynucleotide can be operably linked to a constitutive promoter (e.g., the Rous sarcoma virus long terminal repeat (RSV LTR) promoter/enhancer or the cytomegalovirus major immediate early gene (CMV IE)), an inducible promoter, (e.g., a growth hormone promoter, metallothionein promoter, heat shock protein promoter, E1B promoter, hypoxia induced promoter, radiation inducible promoter, or adenoviral MLP promoter and tripartite leader), an inducible-repressible promoter, or a tissue specific promoter (e.g., a smooth muscle cell α-actin promoter, VEGF receptor promoter, or myosin light-chain 1A promoter). In many instances, host-native promoters are preferred over non-native promoters (e.g., a human α-actin promoter, β-actin promoter, or EF1α promoter linked to a human MxA-encoding sequence may be preferred in a human host), particularly where strict avoidance of gene expression silencing due to host immunological reactions is desirable. Other suitable promoters and principles related to the selection, use, and construction of suitable promoters are provided in, e.g., Werner, Mamm. Genome, 10(2), 168-75 (1999), Walther et al., J. Mol. Med., 74(7), 379-92 (1996), Novina, Trends Genet., 12(9), 351-55 (1996), Hart, Semin. Oncol., 23(1), 154-58 (1996), Gralla, Curr. Opin. Genet. Dev., 6(5), 526-30 (1996), Fassler et al., Methods Enzymol., 273, 3-29 (1996), Ayoubi et al., FASEB J, 10(4), 453-60 (1996), Goldsteine et al., Biotechnol. Annu. Rev., 1, 105-28 (1995), Azizkhan et al., Crit. Rev. Eukaryot. Gene Expr., 3(4), 229-54 (1993), Dynan, Cell, 58(1), 1-4 (1989), Levine, Cell, 59(3), 405-8 (1989), and Berk et al., Annu. Rev. Genet., 20, 45-79 (1986), as well as U.S. Pat. No. 6,194,191. In some aspects, radiation-inducible promoters such as those described in described in U.S. Pat. Nos. 5,571,797, 5,612,318, 5,770,581, 5,817,636, and 6,156,736 can be suitable (such as where administration of the polynucleotide in connection with radiation therapy is sought). In other instances, ecdysone and ecdysone-analog-inducible promoters (ecdysone-analog-inducible promoters are commercially available through Stratagene (LaJolla Calif.)). Other suitable commercially available inducible promoter systems include the inducible Tet-Off or Tet-On systems (Clontech, Palo Alto, Calif.).

The polynucleotide sequence also or alternatively can comprise an upstream activator sequence (UAS), such as a Gal4 activator sequence (as described in, e.g., U.S. Pat. No. 6,133,028) or other suitable upstream regulatory sequence (as described in, e.g., U.S. Pat. No. 6,204,060). The polynucleotide can include any other expression control sequences (e.g., enhancers, termination sequences, initiation sequences, splicing control sequences, etc.). Typically, the polynucleotide will include a Kozak consensus sequence, which can be a naturally occurring or modified sequence such as the modified Kozak consensus sequences described in U.S. Pat. No. 6,107,477. The polynucleotide can further comprise site-specific recombination sites, which can be used to modulate transcription of the polynucleotide, as described in, e.g., U.S. Pat. Nos. 4,959,317, 5,801,030 and 6,063,627, European Patent Application 0 987 326 and International Patent Application WO 97/09439.

The polynucleotide preferably is positioned in and/or administered in the form of a suitable delivery vehicle (i.e., a vector). The vector can be any suitable vector. For example, the nucleic acid can be administered as a naked DNA or RNA vector, including, for example, a linear expression element (as described in, e.g., Sykes and Johnston, Nat. Biotech., 17, 355-59 (1997)), a compacted nucleic acid vector (as described in, e.g., U.S. Pat. No. 6,077,835 and/or International Patent Application WO 00/70087), a plasmid vector such as pBR322, pUC 19/18, or pUC 118/119, a “midge” minimal-sized vector (as described in, e.g., Schakowski et al., Mol. Ther., 3, 793-800 (2001)), or as a precipitated nucleic acid vector construct (e.g., a CaPO4 precipitated construct). The vector also can be a shuttle vector, able to replicate and/or be expressed (desirably both) in both eukaryotic and prokaryotic hosts (e.g., a vector comprising an origin of replication recognized in both eukaryotes and prokaryotes). The nucleic acid vectors of the invention can be associated with salts, carriers (e.g., PEG), formulations which aid in transfection (e.g., sodium phosphate salts, Dextran carriers, iron oxide carriers, or gold bead carriers), and/or other pharmaceutically acceptable carriers, some of which are described herein. Alternatively or additionally, the polynucleotide vector can be associated with one or more transfection-facilitating molecules such as a liposome (preferably a cationic liposome), a transfection facilitating peptide or protein-complex (e.g., a poly(ethylenimine), polylysine, a virus like particle (VLP), or viral protein-nucleic acid complex), a virosome, a modified cell or cell-like structure (e.g., a fusion cell), or a viral vector.

Any suitable viral vector can be used to deliver the polynucleotide. The viral vector can be a vector that requires the presence of another vector or wild-type virus for replication and/or expression (i.e., a helper-dependent virus), such as an adenoviral vector amplicon or adeno-associated virus (AAV) vector. The viral vector can take the form of a wild-type viral particle comprising an insertion of the Mx-encoding nucleic acid. Typically, the viral particle will be modified in its protein and/or nucleic acid content to increase transgene capacity or aid in transfection and/or expression of the nucleic acid (examples of such vectors include the herpes virus/AAV amplicons). Such vectors are typically named for the type of virus they are obtained from, derived from, or based upon, as applicable. Examples of proven viral gene transfer vectors include herpes viral vectors, adeno-associated viral vectors, and adenoviral vectors. Suitable examples of such vectors and other suitable viral vectors are provided in, e.g., Mackett et al., J. Gen. Virol., 67, 2067-82 (1986), Beaud et al., Dev. Biol. Stand., 66, 49-54 (1987), Levine, Microbiol. Sci., 4(8), 245-50 (1987), Lebowski et al., Mol. Cell Biol., 8(10), 3988-96 (1988), Nicholas et al., Biotechnology, 10, 493-513 (1988), Moss et al., Curr. Top. Microbiol. Immunol., 158, 25-38 (1992), Berihoud et al., Curr. Opin. Biotechnol., 10(5), 440-47 (1999), Yonemitsu, Nat. Biotechnol., 18(9), 970-3 (2000), and Russell, J. Gen. Virol., 81, 2573-2604 (2000), as well as International Patent Application WO 00/32754.

The construction of recombinant viral vectors is well understood in the art. For example, adenoviral vectors can be constructed and/or purified using the methods set forth, for example, in Graham et al., Mol. Biotechol., 33(3), 207-220 (1995), U.S. Pat. Nos. 5,922,576, 5,965,358 and 6,168,941 and International Patent Applications WO 98/22588, WO 98/56937, WO 99/15686, WO 99/54441, and WO 00/32754. Adeno-associated viral vectors can be constructed and/or purified using the methods set forth, for example, in U.S. Pat. No. 4,797,368 and Laughlin et al., Gene, 23, 65-73 (1983). Similar techniques are known in the art with respect to other viral vectors, particularly with respect to herpes viral vectors (see e.g., Lachman et al., Curr. Opin. Mol. Ther., 1(5), 622-32 (1999)), lentiviral vectors, and other retroviral vectors.

The viral vector can be a chimeric viral vector, derived from two or more viral genomes. Examples of suitable chimeric viral vectors are described in, e.g., Reynolds et al., Mol. Med. Today, 5(1), 25-31 (1999) and Boursnell et al., Gene, 13, 311-317 (1991).

The viral vector is preferably a replication-deficient viral vector (e.g., an E1, E2, and/or E4 deleted adenoviral vector). Examples of replication deficient adenoviral vectors are disclosed in, for example, U.S. Pat. Nos. 5,851,806, 5,985,655, and 5,994,106 and International Patent Applications WO 95/34671 and WO 97/21826.

A vector, such as particular types of viral vector particles, can integrate into the host cell's genome or be a non-integrative vector. Non-integrative vectors, e.g., naked DNA plasmids, and particularly non-integrative viral vectors (e.g., adenoviral vectors), are typically preferred. Alternatively, a lentiviral vector, naked DNA vector comprising integration-promoting sequences (as described in, e.g., International Patent Applications WO 98/41645 and WO 98/54345), or AAV viral vector that integrates into the host cell's genome at defined locations can be used. Additionally, when using a non-integrating viral vector, control sequences that allow for retention of the delivered transgene in the host cell, either by integration into the target cell genome or by maintenance as an episomal nucleic acid can be utilized (as discussed in, e.g., International Patent Application WO 98/54345).

Adenoviral vectors can be used for short-term or intermediate term expression of the fusion protein in dosages such as those described above. Where longer expression (e.g., about three months, about six months, about nine months or longer) is desired, retroviral vectors (e.g., lentivirus vectors) or adeno-associated viral (AAV) vectors can be advantageously used (as described in, e.g., Buschacher et al., Blood, 5(8), 2499-504, Carter, Contrib. Microbiol., 4, 85-86 (2000), Smith-Arica, Curr. Cardiol. Rep., 3(1), 41-49 (2001), Taj, J. Biomed. Sci., 7(4), 279-91 (2000), Vigna et al., J. Gene Med., 2(5), 308-16 (2000), Klimatcheva et al., Front. Biosci., 4, D481-96 (1999), Lever et al., Biochem. Soc. Trans., 27(6), 841-47 (1999), Snyder, J. Gene Med., 1(3), 166-75 (1999), Gerich et al., Knee Surg. Sports Traumatol. Arthrosc., 5(2), 118-23 (1998), During, Adv. Drug Deliv. Review, 27(1), 83-94 (1997), and U.S. Pat. Nos. 4,797,368, 5,139,941, 5,173,414, 5,614,404, 5,658,785, 5,858,775, and 5,994,136, as well as other references discussed elsewhere herein). Alternatively, polynucleotide vectors can be used, or host integrative techniques can be employed. Pox virus vectors (e.g., MVA vectors), HSV vectors, and alphavirus vectors also can be useful gene delivery vehicles.

The viral vector is preferably a “targeted” vector, comprising one or more modifications that increase or decrease the wild-type tropicity of the vector (e.g., by targeting the vector to particular cancer cells). Manipulation of viral capsid (coat) proteins can broaden the range of cells infected by a viral vector or enable targeting of a viral vector to a specific cell type. Examples of targeted adenoviral vectors in this respect are described in Wickam, Gene Ther., 7(2), 110-14 (2000), in addition to U.S. Pat. Nos. 5,559,099, 5,731,190, 5,712,136, 5,770,442, 5,846,782, 5,962,311, 5,965,541, 5,985,655, 6,030,954, and 6,057,155. In non-viral vector systems, targeting can be accomplished through the use of targeting peptides linked to the polynucleotide sequence (e.g., an asialoorosomucoide protein, which promotes liver cell targeting, can be conjugated to the polynucleotide (as described in, e.g., Wu and Wu, J. Biol. Chem., 263 (29), 14621-24 (1988)). Targeted cationic lipid compositions also can be used to deliver the polynucleotide, such as the compositions described in U.S. Pat. No. 6,120,799.

The Mx proteins and polynucleotides of the invention also are desirably targeted to cancer cells by conjugation with a cancer cell-targeting agent. For example, the Mx protein of the invention can be in the form of a fusion protein comprising a suitable cancer cell-targeting domain. Mx-encoding polynucleotides and polynucleotide vectors can be associated with proteins or other molecules that target cancer cells. The polynucleotide or protein can, for example, be conjugated to an antibody directed to a particular cancer cell antigen. PEGylation of the Mx protein also can result in cancer cell targeting.

The vector is desirably administered such that immune response to the vector is minimized. For example, minimization of immune response against an adenoviral can be obtained through the methods described in U.S. Pat. Nos. 6,093,699 and 6,211,160, U.S. Patent Application 2001-0006947A1, and International Patent Applications WO 98/40509 and WO 00/34496.

Alternatively, the nucleic acid can be positioned in, and delivered via, a transformed host cell (i.e., in an ex vivo gene therapy method), which is delivered near or in the cancer such that Mx expressed from the Mx-encoded polynucleotide results in a therapeutic effect (e.g., reduction of cancer progression). Suitable transformed cells can be obtained by contacting a cell suitable for delivery and expression of the Mx-encoding nucleic acid in or near the cancer (e.g., a cell removed from the host) with an Mx-encoding polynucleotide (or vector) such that transformation (either integrative or non-integrative) occurs. The cells are implanted (or re-implanted, as the case may be) in or near the cancer. Guidance in performing ex vivo gene therapy techniques is provided in the techniques described in, e.g., Crystal et al., Cancer Chemother. Pharmacol., 43(Suppl.), S90-S99 (1999) and U.S. Pat. Nos. 5,399,346 and 6,180,097.

The administration of the Mx protein and/or Mx-encoding polynucleotide to a host according to the invention desirably reduces the growth rate of the cancer cells, reduces the metastatic potential of the cancer cells, or both. The reduction of “metastatic potential,” i.e., the probability that a cancer (population of cancer cells) will metastasize, is particularly preferred. The reduction of metastatic potential can be determined using any suitable technique. Several techniques are known in the art. Examples of suitable techniques for assessing the metastatic potential of a population of cancer cells include the techniques described in U.S. Pat. Nos. 5,536,642, 5,643,557, 5,688,694, 5,753,437, 5,869,238, 6,228,345, and references cited therein, in addition to, e.g., Radinsky et al., Clin. Cancer Res., 1(1), 19-31 (1995), Mittleman et al., Biochem. Biophys. Res. Commun., 203(2), 899-906 (1994), Yabkowitz et al., Cancer Res., 53(2), 378-87 (1993), Carter et al., J. Urol., 142(5), 1338-41 (1989), Partin et al., Proc. Natl. Acad. Sci. USA, 86(4), 1254-8 (1989), Ochalek et al., Cancer Res., 48(16), 5124-8 (1988), and Price, J. Natl. Cancer Inst., 77(2), 529-35 (1986). A preferred technique for assessing metastatic potential is the hepatic metastasis assay, which is a recognized model for predicting reduction in cancer progression in vivo (see, e.g., Hashino et al., Clin. Exp. Metastasis, 121(4), 324-8 (1994) and Jessup et al, Br. J. Cancer, 67(3), 464-70 (1993)).

Reduction of tumor growth in the context of the present invention comprises any detectable reduction in the rate of growth of at least one tumor. Tumor growth, and thus the reduction thereof, can be determined using any suitable technique, including several of the above-described techniques for detecting cancer (e.g., biopsy and PET). Other suitable techniques for assessing tumor growth are set forth in, e.g., Tubiana, Acta Oncol., 28(1), 113-21 (1989), Miller et al., Toxicology, 145(2-3), 115-25 (2000), Takiguchi et al., Clin. Exp. Metastasis, 13(3), 184-90 (1995), Bassukas, Anticancer Res., 13(5A), 1601-6 (1993), Orr et al., Clin. Exp. Metastasis, 4(2), 105-16 (1986), Laing et al., J. Natl. Cancer Inst., 51(4), 1345-8 (1973), Coons et al., Cancer, 19(9), 1200-4 (1966), Ryggard et al., Breast Cancer Res. Treat., 46(2-3), 303-12 (1997), and Kuroisi et al., Jpn. J. Cancer Res., 81(5), 454-62 (1990). A preferred assay for assessing tumor growth is the primary tumor growth assay, which is recognized as a useful model in the art (see, e.g., Yan et al., Eur. J. Cancer, 36(2), 221-8 (2000) and Lin et al., J. Surg. Oncol., 63(2), 112-8 (1996)). Administration or Mx polypeptides and/or Mx-encoding nucleic acids in accordance with the invention advantageously can reduce or halt tumor growth or even reduce tumor size in vivo in a mammalian host (e.g., a human cancer patient).

In another aspect, the invention provides a method of reducing cancer progression comprising increasing the level of an Mx in a population of cancer cells, such that the growth rate of the cancer is reduced, the metastatic potential of the cancer is reduced, or both. The level of the Mx in the population of cancer cells can be increased by any suitable technique, including, e.g., administration of an Mx-encoding polynucleotide, administration of an Mx, or administration of a factor which upregulates or down-regulates Mx expression (e.g., a virus, viral protein, collection of viral proteins, viral nucleic acid, viral nucleic acid-derived nucleic acid, a collection of such viral and/or viral-derived nucleic acids, or a combination of any thereof, which induce Mx expression in or near the cells). The cells targeted by such therapeutic techniques can be any suitable target cells. In a particular aspect, the cells have normal physiological levels of type-1 interferons (IFN-α and IFN-β) and IFN-γ (associated with such cells). As such, the cells in such particular aspects are free of exogenous type 1 IFN, IFN-γ, or nucleic acid encoding IFN-α, IFN-β, or IFN-γ that might induce MY-expression prior to and during the method. In other aspects, the invention provides a method of reducing cancer progression comprising administering an Mx, an Mx-encoding nucleic acid, a recombinantly modified cell that overexpresses an Mx, a recombinant cell that comprises one or more recombinant and typically heterologous Mx-encoding nucleic acids, to a group of cancer cells (e.g., one or more tumors) that express irregular levels of type-1 interferons (e.g., cells that overexpress IFN-α and/or IFN-β). Indeed, the therapeutic methods of the invention may be particularly useful in cells that overexpress one or more type-1 interferons. Assays for IFNs are known in the art (see, e.g., McNeil et al, J. Immunol. Methods, 46(2), 121-7 (1981), Green et al., Tex. Rep. Biol. Med., 35, 167-72 (1977), and Finter, Tex Rep. Biol. Med., 35, 161-6 (1977)). The target cells can be associated with any level of Mx expression. Thus, the invention provides a method of administering an Mx, an Mx-encoding nucleic acid, or associated cell or vector to a group of target cells that lack any detectable level of Mx expression, that are characterized by reduced Mx expression levels, or that have normal (or even above normal) levels of Mx expression. Thus, the therapeutic methods of administering an Mx, Mx-encoding nucleic acid, and/or Mx expression-inducing molecule can act in conjunction with the ability of a host's cells to express an endogenous, wild-type Mx (e.g., human MxA). Therefore, for example, the methods of the invention can be used to reduce the mobility of Mx-expressing cancer cells in a host afflicted with a cancer.

The delivery of an Mx protein, Mx-encoding polynucleotide, or both, desirably reduces the motility of the cancer cells. The reduction of motility resulting from delivery or expression of the Mx protein in or near the cancer cells can be by any detectable amount of reduction. The artisan will typically assess the reduction of motility associated with the method of the invention by comparing the motility of the cancer cells to a control host or predictive model that does not receive the Mx, Mx-encoding polynucleotide, or combination thereof. Motility can be specifically measured using any suitable techniques. Examples of suitable techniques are described in, e.g., Gildea et al., Biotechniques, 29(1), 81-6 (2000), Tatsuka et al., Jpn. J. Cancer Res., 80(5), 408-12 (1989), Benestad et al., Cell Tissue Kinet., 20(1), 109-19 (1987), Aroskar et al., Tumori, 72(3), 225-29 (1986), and Perrot et al., int. J. Cell Cloning, 3(1), 33-43 (1985). A preferred measure of motility is the Boyden chamber cell motility assay, which is described in, e.g., Taraboletti et al., J. Cell Biol., 105(5), 2409-15 (1987) and U.S. Pat. Nos. 6,150,117 and 6,177,244.

As discussed above, the method of the invention can be applied to cells in vitro or in vivo. Preferably, the method is applied where the cancer cells are in a vertebrate, which desirably is a mammal. Most preferably, the cancer cells are in a human.

The invention further provides a method of assessing the metastatic potential of a cancer by obtaining a sample of the cancer, determining the level of Mx, Mx-encoding nucleic acid, or both in the sample, and assessing the metastatic potential of the cancer by comparing the level of Mx, Mx-encoding nucleic acid, or both, with a control. The cancer in this respect can be any suitable cancer, such as the cancers discussed elsewhere herein. A sample of the cancer can be obtained using conventional techniques (e.g., biopsy).

The amount of Mx in the cancer sample can be determined using any suitable technique, examples of which are provided in references discussed above (e.g., by using antibody-binding assays or direct quantification of Mx protein levels). Other general methods of determining protein levels include Western blot techniques (as described in, e.g., U.S. Pat. Nos. 4,452,901 and 5,356,772), ELISA techniques (as described in, e.g., Abe et al., Clinica Chimica Acta, 168, 87-95 (1987), and the Lowry colorimetric protein assay (see, e.g., Lowry et al., J. Biol. Chem., 193, 265-75 (1951)). The level of Mx-encoding polynucleotide can be determined by any suitable technique. Levels of RNA expression in the cancer sample can be determined by Northern Blot analysis (discussed in, e.g., McMaster et al., Proc. Natl. Acad. Sci. USA, 74, 4835-38 (1977) and Sambrook et al., supra), RT-PCR (as described in, e.g., U.S. Pat. No. 5,601,820 and Zaheer et al., Neurochem. Res., 20, 1457-63 (1995), and in situ hybridization techniques (as described in, e.g., U.S. Pat. Nos. 5,750,340 and 5,506,098). Because Mx proteins are associated with strong transcriptional regulation, RT-PCR and other nucleic acid techniques often can be correlated to the amount of Mx present in a sample.

The control can be any suitable control. Typically, a control will be a similar cell (e.g., morphologically and genetically similar to the cancer cell) that comprises a known amount of Mx, expresses an Mx at a known level, or a combination thereof. In other instances, the control can be a model, such as an amount of Mx or a level of Mx gene expression that, based on earlier experimentation and analysis, is correlated with an increase or decrease in metastatic potential.

The inventors have discovered that the amount of Mx, Mx-encoding nucleic acid, or both, in a population cancer cells can be correlated with metastatic potential of the cells. In general, a decreased level of Mx, Mx-encoding nucleic acid, or both in the cell sample as compared to the control is indicative of an increased potential for metastasis, and an increased level of Mx, Mx-encoding nucleic acid, or both in the cell sample as compared to the control is indicative of a decreased potential for metastasis. A lack of change or difference in the cancer cell sample with respect to the control indicates no change in metastatic potential. In a preferred aspect, a cancer sample is obtained from a mammalian host and the method further comprises prognosticating the likelihood of survival of the mammal. The control for such a method will preferably comprise information correlating the measured metastatic potential, determined by the amount of Mx and/or Mx-encoding nucleic acid in the cancer sample, with the likelihood of survival of the host. Such correlations can be made through the exercise of routine experimentation.

The results of the above-described assay can be used as an indicator for assessing if delivery of an anti-cancer therapeutic composition and/or application of an anti-cancer therapeutic technique (e.g., radiation therapy, chemotherapy, or surgery) is appropriate. Where the level of Mx, Mx-encoding nucleic acid, or both, is lower than the level of the control, the method typically further comprises delivering to the cancer an agent that reduces the metastatic potential of the cancer. The agent can be any suitable agent. For example, the agent can be a small molecule pharmaceutical (e.g., an alkylating agent such as Melphalan, Paclitaxel (Taxol), or zoladex) or a polynucleotide encoding a protein with anti-cancer activity (e.g., a tumor suppressor gene, such as p53, a tumor necrosis factor (e.g., TNF-α), or a cancer-specific antigen (e.g., CEA, KSA, or PSA)). Examples of suitable anti-cancer agents are described in U.S. Pat. No. 6,235,761. In a preferred method, the agent comprises a therapeutic amount of an Mx (most preferably MxA), a therapeutic amount of a nucleic acid encoding an Mx, or both.

In another aspect, the invention provides a method of assessing the ability of an agent to affect the level of expression of an Mx (typically an exogenous Mx such as MxA). A cell expressing a known level of an Mx is obtained, contacted with a non-IFN agent to be tested, and the cell is thereafter assayed for expression of the Mx to assess the ability of the non-IFS agent to affect the level of Mx expression. The agent can be any agent, preferably an agent other than an IFN, which can be delivered to the cell. The detection of Mx expression can be accomplished using any suitable technique, such as techniques for detecting gene expression discussed elsewhere herein. Detection of a decreased level of expression of Mx in the cell upon administration of the agent will desirably be correlated to the carcinogenicity of the agent and/or tumorigenicity of the agent.

In a related aspect, the invention provides a method of assessing the ability of an agent to affect the level of activity of an Mx promoter or other Mx nucleic acid regulatory sequences. The method comprises obtaining an Mx promoter and linking the promoter to a suitable reporter gene to form a reporter construct. The reporter gene can be any nucleic acid sequence that, when expressed, produces a readily assayable protein. The reporter gene can be any suitable reporter gene. Several types of reporter genes are known. Examples of well-characterized suitable reporter genes include β-Gal genes chloramphenicol acetyltransferase (CAT) genes, β-glucoronidase, firefly luciferase, and green fluorescent protein (GFP) genes. A suitable cell is transformed with the reporter gene construct. Illustrative suitable cell lines include, for example, NIH 3T-3, MDA, MD-MB231, and osteoclasts. The method comprises contacting the cell with a non-IFN, non-viral agent and assaying for the level of reporter gene expression. In another aspect, the method is performed with a viral agent (e.g., a particular segment of viral RNA). In general, agents that decrease reporter gene expression are expected to be associated with increased cancer progression due to the down regulation of the associated Mx, which typically is an endogenous wild-type Mx (e.g., MxA). Changes in reporter gene expression can be determined by any suitable technique (e.g., by detecting increase of reporter gene mRNA levels by a Northern Blot and/or by detecting levels of expressed protein by a Western blot or promoter/reporter chemiluminescence). A preferred aspect of the method comprises identifying agents that are carcinogenic and/or tumorigenic by determining that such agents downregulate the activity of the MxA promoter.

In another aspect, other portions of the MxA gene (or other Mx gene) are linked to a reporter and similarly screened for downregulation of MxA expression or MxA gene activity. For example, particular domain-encoding regions or the region upstream of the MxA promoter (SEQ ID NO:5) can be linked to suitable reporter constructs for assessing whether particular agents (e.g., natural, semisynthetic, or synthetic small molecule compounds—usually nonpolypeptide and nonpolynucleotide organic molecules) target these regions of the MxA gene and/or whether particular sequence mutations/modifications modulate the biological activity of the Mx protein and/or gene.

In a further aspect, the invention provides a method of assessing the metastatic potential of a cancer in a host comprising obtaining a sample of the cancer and assessing the metastatic potential of the cancer by determining the level of expression of at least one mutant Mx in the cells of the cancer. Methods for determining levels of gene expression are described elsewhere herein. Briefly, a probe comprising the cDNA sequence or genomic DNA sequence of an Mx can be used to screen for related polynucleotides as described elsewhere herein (such screening methods are well known—see, e.g., Sambrook, supra). Through sequence analysis, identified sequences that bind to the probe or probes can be evaluated to determine whether a mutant Mx gene is in the analyzed cells. The method can be performed with any suitable mutant Mx protein. Typically, the Mx mutant will exhibit a reduced GTPase activity, reduced tubulin association, or both in the sample as compared with wild-type Mx expressed in a non-cancerous cell of the host. The mutant Mx also or additionally can lack other biological functions associated with its non-mutant wild-type Mx counterpart, including, e.g., a reduction or reduction in oligomer formation. Techniques for assessing GTPase activity and tubulin association are described elsewhere herein. The mutant can comprise any number of amino acid substitutions, additions, or deletions, with respect to its wild-type counterpart. The mutant can comprise any number of such mutations in any domain of the Mx protein. Typically, the mutant will comprise at least one amino acid substitution, addition, or deletion in the Mx dynamin GTPase domain, Mx dynamin central domain, Mx LZ1 domain, and/or the Mx GTPase effector domain. For example, the method can comprise determining the level of expression of a mutant Mx comprising a mutation in the dynamin GTPase domain, such as a deletion of the N-terminal most threonine residue of the dynamin GTPase domain normally found in the Mx (e.g., Thr103 in the case of human MxA). Such mutants are known or believed to be associated with increased metastatic potential. Similar to other aspects, the assessment that such an Mx is expressed in the cell can be combined with a suitable anti-cancer treatment (e.g., TNF-α gene therapy), which can include administration of therapeutic amount of an Mx, Mx-encoding nucleic acid, or both, wherein the Mx, or Mx-encoding nucleic acid, or both, exhibit at least non-cancerous wild-type levels of GTPase activity and/or tubulin-association. MxA GTPase activity is not limited to the GTPase domain, and accordingly, methods for identifying regions important to a particular Mx-associated biological activity will not be limited to these domains. Assays for determining qualities and properties of the Mx protein to be administered in this respect are described elsewhere herein.

An Mx protein and/or Mx-encoding polynucleotide (or polynucleotide containing vector or cells) can be combined with a pharmaceutically acceptable carrier for use in the therapeutic methods of the invention, and such pharmaceutical compositions form an important aspect of the invention. The term “pharmaceutically acceptable” means that the composition is a non-toxic material that does not interfere with the effectiveness of the biological activity of the Mx and/or other effective ingredients. Any suitable carrier can be used, and several carriers for administration of therapeutic proteins are known in the art. The composition comprising the Mx, Mx-encoding polynucleotide, or both, also can include diluents, fillers, salts, buffers, detergents (e.g., a nonionic detergent), stabilizers, solubilizers, and/or other materials suitable for inclusion in a pharmaceutically composition. It is preferred that the pharmaceutically acceptable carrier be one which is chemically inert to the active compounds and one which has no detrimental side effects or toxicity under the conditions of use. The pharmaceutically acceptable carrier(s) can include polymers and polymer matrices. The choice of carrier will be determined in part by the particular method used to administer the composition. Accordingly, there is a wide variety of suitable formulations of the pharmaceutical composition of the present invention. The pharmaceutical composition of the invention also can contain preservatives, antioxidants, or other additives known to those of skill in the art. Examples of suitable components of the pharmaceutical composition in this respect are described in, e.g., Urquhart et al., Lancet, 16, 367 (1980), Lieberman et al., Pharmaceutical Dosage Forms—Disperse Systems (2nd ed., vol. 3, 1998), Ansel et al., Pharmaceutical Dosage Forms & Drug Delivery Systems (7th ed. 2000), Remington's Pharmaceutical Sciences, Berge et al., J. Pharm. Sci., 66(1), 1-19 (1977), Wang and Hanson, J. Parenteral. Sci. Tech., 42, S4-S6 (1988), U.S. Pat. Nos. 5,708,025, 5,994,106, 6,165,779, 6,225,289, and 6,235,761. The composition will desirably comprise an effective dose of the Mx, Mx-encoding polynucleotide, or both. An effective dose will depend on the desired use of the Mx and/or Mx-encoding polynucleotide, as well as the features of the Mx. General principles in dosing decisions are described in, e.g., Platt, Clin. Lab Med., 7, 289-99 (1987), and in “Drug Dosage,” J. Kans. Med. Soc., 70(1), 30-32 (1969).

In a particular aspect of the invention pertaining to cancer therapy, a therapeutically effective amount of a MxA or MxA homolog is administered to a patient in a suitable form (e.g., in a viral or nonviral vector) having a detectable cancer growth in a discrete area (e.g., the prostrate or breast), preferably in a targeted area around the locus of the cancer and in channels of the body susceptible to cancer spread from the locus of the cancer, so as to detectably reduce, preferably substantially reduce, and more preferably essentially prevent metastasis of the cancer. The method desirably further includes treatment of the exogenous MxA-localized cancer by, for examples, surgery, radiation therapy, chemotherapy, treatment with a cancer vaccine (or associated vector), passive cancer antigen antibody immunization, treatment with oncolytic virus, gene therapy treatment, or other suitable antitumor therapeutic technique. A therapeutically effective dose is any dose that has a detectable therapeutic effect (e.g., a detectable reduction in the size of one or more tumors, the reduction of cancer progression, and/or a reduction in the rate of cancer metastasis) when the dose is administered to a host (e.g., a human cancer patient). Principles for determining therapeutically effective doses (or other suitable doses, such as a prophylactically effective dose) are described herein and/or known in the art with respect to the various types of compositions that can be used in the therapeutic methods of the invention.

In an additional aspect, the invention provides a cell and cell line stably transformed with a nucleic acid comprising a Mx promoter and/or other Mx regulatory sequence, preferably wild-type sequences (e.g., the human MxA promoter or the promoter of the MxA homolog Mx1), operably linked to a reporter gene (e.g., a luciferase gene). Such cells and lines can be used to screen potential regulators of Mx promoter activity.

The invention further provides a method of reducing cancer progression by, for example, administering an effective amount of one or more molecules that increase the expression of an Mx (typically, but not necessarily, an endogenous, wild-type Mx such as human MxA) in a population of cells (typically and preferably in a host), such that the level of Mx expression is upregulated in such cells. In one aspect, the method can be practiced with a viral RNA, viral RNA-derived DNA, or other related nucleic acid (e.g., a modified RNA having improved stability as opposed to a wild-type RNA but comprising components of the viral RNA sequence responsible for the upregulation of MxA) and/or a viral protein. Viral RNAs that activate Mx expression include viruses of the Orthomyxoviridae (e.g., Thogoto virus—THOV), Paramyxoviridae, Rhabdoviridae, Bunyviridae, and Togaviridae families (see, e.g., Hefti et al., J. Virol. 73(8):6984-6991 (1999) for related discussion). Numerous small viral RNA and viral RNA-derived nucleic acids (e.g., viral RNA oligonucleotides or short polynucleotides of about 20, about 50, about 100, about 200, about 300, or more bases in length) are expected to upregulate MxA and other MxA in this respect. Modified and homologous nucleic acids having about 90% identity or more to such viral sequences, identified as regulating Mx expression using the methods provided herein, also can be useful in such a method, as can Mx expression-inducing nonpolypeptide and nonpolynucleotide small molecule compounds identified by such screening techniques.

The following examples farther illustrate the invention but, of course, should not be construed as in any way limiting its scope.

### EXAMPLE 1

This example describes representative experiments that confirm the inventors' discovery that uninduced MxA is expressed in nonmetastatic cells but is undetectable in highly metastatic cancer cells.

Differential display-reverse transcription-polymerase chain reaction (DD-RT-PCR (described in, e.g., Liang et al., Science 257:867-971 (1992)) analysis was performed using 1 μg of poly(A)+ RNA samples obtained from cells of the PC-3 human prostate cancer cell line (ATCC, Manassas, Va.) and the PC-3M cell line, which is a highly metastatic subline derivative of PC-3 (J. Kozlowski, Northwestern University Medical School—see Kozlowski et al., Cancer Res. 44:3522-3529 (1984) for description). Specifically, cDNAs were generated from the RNA samples using Superscript reverse transcriptase (GIBCO BRL—Gaithersburg, Md.) with anchored and arbitrary primers (Operon Biotech—Alameda, Calif.). Differentially expressed bands ranging from 170 to 500 base pairs (bps) in size were nick-translated and used to probe blots containing PC-3 and PC-3M poly(A)+ mRNA.

Eight cDNA fragments with possible differences in expression between PC-3 and PC-3M cells were identified. Northern blot analysis was performed using standard techniques (as described in Sambrook et al., supra). Briefly, 10 μg total RNA from cell pellets of PC-3 and PC-3M cells, separated on a denaturing gel (1% agarose, 20 mM MOPS, 5 mM Na Acetate, 1 mM EDTA, 1.8% formaldehyde, pH 7.0) and blotted on a nylon membrane (HyBondN, Amersham Biosciences—Piscataway, N.J.). Probing the blot with a 32P-labeled nick-translated DD-2 fragment and other MxA cDNA inserts indicated that only one of these eight bands, a 200 base pair (bp) DD-RT-PCR band (DD-2), was differentially expressed, as a strong 3.0 kb mRNA band in PC-3 cells, but not in PC-3M cells.

Sequencing of DD-2 and comparison against the MxA sequence reported in the GenBank sequence database (Accession No. M33882) and the published inferred sequence reported in Horisberger et al., J. Virol. 66:4705-4709 (1990)) revealed that the sequence of DD-2 had a strong resemblance to a portion of the mRNA encoding the interferon-inducible GTPase MxA (two apparently functionally non-significant conservative mutations, one at nt 1378 that resulted in a conservative amino acid change and another silent CCA to GCG mutation at nt 541 (corresponding to nt 556 of GenBank Accession No. M33882), were detected in DD-2 as compared to the previously characterized MxA sequence reported in GenBank). A nearly full-length cDNA clone of DD-2, obtained by screening a PC-3 cDNA library with the DD-2 cDNA probe, was isolated and sequenced and found to contain approximately 70% of the expected 3.0 kilobase (kb) MxA sequence (including 95% of the coding region). Northern blots probed with the isolated MxA clone and a previously characterized clone (see Horisberger et al., 1990, supra), resulted in similar patterns of expression (i.e., abundant expression in PC-3 cells but no detectable expression in PC-3M cells). To ensure equal loading, the test blots were hybridized with a 1.3-kb PstI fragment of rat glyceraldehyde phosphate dehydrogenase (GAPDH) (Fort et al., Nucl. Acids Res. 13:1431-1442 (1985)).

Western blot analysis performed using anti-MxA monoclonal antibody (Horisberger and Hochkeppel, J. Interferon Res. 7:331-343 (1987)) and standard techniques (with 80 μg of cell lysate) corroborated the above-described Northern blot expression data, demonstrating the presence of a 78-kDa protein in PC-3 lysates but not in PC-3M lysates. The Western blot also was probed with anti-tubulin antibody (Oncogene Research Products—San Diego, Calif.) to ensure that the samples were equally loaded.

The above-described MxA cDNA clone and the original MxA cDNA clone were used to analyze MxA expression in normal and tumor cell lines. PC-3 cells exhibited abundant MxA expression, while MxA mRNA was undetectable in PC-3M cells. Western blots confirmed that MxA was present in PC-3 cells but not PC-3M cells.

It was unexpected that PC-3 cells would express MxA spontaneously, as it was previously believed that MxA is not expressed in normal or neoplastic cells in the absence of viral infection or exposure to endogenous interferon (see, e.g., Goetschy et al., J. Virol. 63:2616-2622 (1989) and al-Masri et al., Mol. Pathol., 50:9-14 (1997); but compare Scherf et al., Nat. Genet., 24:236-244 (2000)).

The test PC-3 and PC-3M cells were further evaluated for IFN-α expression levels (using anti-IFN-α antibodies) and no detectable difference was observed.

Genomic DNA from PC-3 and PC-3M cells were digested with EcoRI, BamHI, or Pst1, electrophoretically separated on a Tris Acetate EDTA 1% agarose gel (Sambrook et al., 1989, supra), which was subsequently subjected to Southern blot analysis with a 32P-labeled nick-translated insert from a previously characterized full-length MxA cDNA (Horisberger et al., J. Virol. 66:4705-4709 (1990)). PC-3 and PC-3M genomic DNA showed identical patterns of hybridization to the MxA probe.

PC-3 and PC-3M test cells also were treated with recombinant IFN-α (Novartis Pharma—Basel, Switzerland) (1000 IU of IFN-α/mL), grown for 24 hours, fixed, permeabilized, and subjected to immunohistochemical analysis using anti-MxA antibody and DAPI nuclear counterstaining to locate individual cells. Consistent with the Western blot results, this assay detected MxA protein only in the untreated PC-3 cells and not in the untreated PC-3M cells (cells were observed with a Zeiss Axiophot microscope with a 40× objective and the images were captured on an Optronics CCD camera). After exposure to IFN-α, the level of MxA protein increased substantially in the PC-3 cells, while MxA protein became detectable for the first tine in the PC-3M cells. Western blotting with sheep anti-IFN-α globulin using 100 μg cell lysates confirmed IFN-α-induced increase in MxA expression in both cell lines.

The PC-3 and PC-3M cells used in this Experiment and the other Experiments described herein were cultured using previously described techniques (see, e.g., Lee et al., J. Biol. Chem. 273:10618-10623 (1998)), unless otherwise stated.

The results of these experiments serve to establish that MxA expression is at least substantially reduced in metastatic PC-3M cells as compared to less metastatic PC-3 cells. The above-described experiments also confirmed that this difference in MxA expression was observable at both the RNA and protein levels; was not the result of genomic deletion or rearrangement; was not due to a difference in IFN-α expression levels; and was not due to a difference in the ability of these cells to respond to IFN-αstimulation of MxA expression.

### EXAMPLE 2

This example demonstrates that cancer cells expressing MxA have a reduced metastatic potential as compared to cancer cells that do not express MxA.

The PC-3M cells of Example 1 were transfected with a pCIneo plasmid (Promega, Madison, Wis.) expressing full-length MxA or a plasmid constructed from pHβ Apr-1 comprising an MxA sequence operably linked to a CMV promoter, and two stable cell lines were selected. PC-3M cells stably transfected with a plasmid expressing β-galactosidase (β-gal) were used as a control. MxA expression was not detected in the PC-3M β-gal cells. The highly metastatic melanoma cell line LOX (ATCC—Manassas, Va.; Dr. Dan Sackett—NICHD; see Fodstad et al., Int. J. Cancer, 41:442-449 (1988)), which does not express endogenous MxA, also was stably transfected with a FLAG-tagged pCIneo plasmid expressing full-length MxA and a FLAG-tagged βgal plasmid.

The motility of PC-3M transfectants in vitro was tested by measuring the ability of the cells to migrate through pores in a membrane. Briefly, FALCON cell culture inserts with an 8-μm pore-size PET membrane (Fisher Scientific, Franklin Lake, N.J.) were placed into the wells of a 24-well plate, each well containing 0.5 ml of complete medium (RPMI 1640 with 10% FBS, 1% antimycotic-antibiotic solution, and 500 μg mL⁻¹ G418). Control and MxA-transfected cells were trypsinized, suspended at 1.5×10⁵ cells/ml in complete medium, and 350 μl of the cell suspension was added to each insert. Following incubation for 24 hours at 37° C., cells from the upper surface of the membrane were removed by scrubbing with a cotton swab. Cells that had migrated through the insert and adhered to the bottom of the membrane were Wright stained using the CAMCO Quik Stain kit (Fisher Scientific), visualized using a Zeiss Axiophot microscope or Leica DMIRB microscope, and counted. MxA expression inhibited the motility of PC-3M cells to levels as low as 24.3% of the β-gal control cells, and for all MxA-expressing cells at least 22.4% lower than the β-gal control cells in both cases tested; and the level of inhibition correlated with the level of exogenous MxA expression.

The motility and invasiveness of the LOX transfectants in vitro was tested using the method described above, except that BIOCOAT Matrigel invasion chamber inserts (Becton Dickinson—Franklin Lakes, N.J.) were used instead of FALCON inserts. MxA expression in LOX cells inhibited the in vitro invasive activity of LOX cells to 15.1% of the invasiveness exhibited by control cells.

### EXAMPLE 3

This example demonstrates that cancer cells expressing MxA develop tumors at a slower rate as compared to cancer cells that do not express MxA.

The effect of MxA expression on tumor growth in vivo was first tested using a primary tumor growth assay. 2×10⁶ recombinant PC-3M cells expressing endogenous MxA (PC3M-MxA) or 2×10⁶ recombinant PC-3M-β-gal cells (expressing β-gal) were injected subcutaneously into 30 beige/SCID mice (Charles River Laboratories, Wilmington, Mass.), and the time to formation of a 2-cm subcutaneous tumor (as measured using Vernier calipers) was determined by monitoring the mice at least three times weekly for evidence of tumor development, quantification of tumor size, and evidence of tumor/metastasis-associated morbidity. Subcutaneous tumor size was quantified using Vernier Calipers. Criteria for sacrifice included tumor growth to greater than 2.0 cm, ill thrift, anorexia, dehydration, decreased activity and grooming behavior, and dyspnea.

The time to develop a 2-cm tumor mass in mice receiving PC3M-MxA cells (on average, 46.8±9.9 days to tumor) was longer than in mice receiving the same number of PC-3M-β-gal cells (on average, 29.8±3.4 days to tumor), although the number of tumors in each group of mice was similar.

The in vivo effects of MxA expression on tumor growth and metastatic potential was also tested using an experimental hepatic metastasis assay. Briefly, 2×10⁶ cells from the PC3M-MxA cell line and the PC-3M-β-gal cell line were injected into the spleens of beige/SCID mice. Mice were monitored at least three times weekly for evidence of tumor development, quantification of tumor size, and evidence of tumor and/or metastasis-associated morbidity. Animal survival time was determined, and mean survival time was compared between the two treatment groups. Liver metastases were observed in both groups at the time of death or sacrifice. The metastases of PC-3M-β-gal cells occurred earlier and resulted in more rapid metastasis-associated morbidity than PC3M-MxA cells (23.3±3.3 days and 54.3±11.2 days survival, respectively).

The results of these experiments confirm that Mx proteins, and particularly endogenous MxA, slows the development of metastases and mitigates aspects of tumorigenesis in vivo. In other words, these data demonstrate a role for MxA as an inhibitor of tumor progression and tumor metastasis in vivo. This experiment also demonstrates that ex vivo administration of recombinant MxA-expressing cells can reduce cancer metastasis in vivo.

### EXAMPLE 4

This example demonstrates that an MxA lacking the N-terminal most threonine residue of the dynamin/self-assembly region of the GTPase domain of endogenous MxA eliminates the ability of MxA to reduce the metastatic potential of a cancer cell and does not bind tubulin.

The threonine 103 residue of MxA in the FLAG-tagged MxA plasmid of Example 2 was mutated to an alanine residue via site-directed mutagenesis. This mutation inactivates MxA GTPase activity. LOX cells were stably transfected with the FLAG-tagged MxA T103 mutant plasmid (T103 MxA-LOX). This point mutation in the GTPase domain resulted in an in vitro invasive activity that was 119% of the β-gal control LOX cells, as quantified using the method described in Example 2.

MxA-LOX cells and T103 MxA-LOX cells were subjected to immunological analysis using known techniques (see Choi et al., J. Biol. Chem., 272:28479-28484 (1997)) with an anti-FLAG antibody after cytoskeletal extraction, which removed all the soluble proteins, leaving only cytoskeleton-associated proteins. Cytoskeletal preparations were prepared by permeabilizing the cells with 1% Triton X-100 in PHEM buffer (60 mM piperazine-N-N′-bis(2-ethane-sulfonic acid) (PIPES; pH 6.9), 25 mM N-2-hydroxyethlypiperazine-N′-2-ethanesulfonic acid (HEPES), 2 mM MgCl₂, and 10 mM ethylene glycol-bis(b-aminoethyl ether)-N,N,N′N′-tetraacetic acid (EGTA, pH 6.9)) for 2 minutes, fixing the cells with 3.7% formaldehyde for 10 minutes at room temperature (see Hartwig, J. Cell Biol. 118:1421-1442 (1992)). Fixed cells were incubated with appropriate primary and secondary antibodies and the nuclei counterstained with 4,6-daimidino-2-phenylinodole (DAPI). Cells were visualized with a Zeiss Axiophot microscope and images were captured using an Optronics CCD camera. Wild-type MxA protein remained bound to the insoluble cytoskeletal matrix, while the T103 mutant MxA protein was undetectable after cytoskeletal extraction.

Coimmunoprecipitation of wild type and mutant MxA-LOX cell lines with anti-α-tubulin (Oncogene Research Products—San Diego, Calif.) and affinity-purified anti-MxA polyclonal antibodies (Yamada et al., Neurosci. Lett. 181:61-64 (1994)), confirmed that the T103 MxA mutant did not associate with cytoskeletal proteins such as tubulin whereas non-mutant MxA associated with cell microtubules and particularly tubulin. In contrast, endogenous MxA did not co-immunoprecipitate with actin in PC-3 cells. Immunoprecipitation was performed by known techniques (Bang et al., Proc. Natl. Acad. Sci. USA 91:5330-5334 (1994)). Briefly, cell lysates were obtained and incubated with the indicated antibodies overnight at 4° C. The immunocomplex was immobilized on protein A/G-Sepharose (Santa Cruz Biotechnologies), resolved on SDS-polyacrylamide gels, transferred to nitrocellulose filters, and immunoblotted with the indicated antibodies.

This example demonstrates that the GTPase domain, specifically the previously characterized dynamin/self-assembly region of the MxA GTPase domain (several regions are now reportedly involved in self-assembly), has a functional role in the ability of WA to inhibit invasiveness of metastatic cancer cells and for WA to associate with microtubules and tubulin. This example also demonstrates strategies by which MxA variants can be screened for biological activity. Although this example demonstrates such screening in terms of the GTPase domain, the method can be similarly applied to other regions involved in WA biological function, including other regions of MxA that are involved in GTPase and/or self-assembly outside of the above-described domains (see, e.g., Janzen et al., J. Virol 74:8202-8206 (2000) for a description of such a GTPase-inactivating mutant and Kochs et al., J. Biol. Chem. 277(16):14172-14176 (2002) concerning the complexity of the GTPase and self-assembly functions of HA).

### EXAMPLE 5

This example further demonstrates that MxA associates with microtubules.

Using the LOX cells stably transfected with the endogenous MxA construct, as described in the preceding Examples, whole cell lysates were prepared and immunoprecipitated with anti-α-tubulin antibodies, anti-MxA antibodies, and protein A/protein G-coated Sepharose beads alone, and the resulting compositions were subjected to Western blotting with anti-FLAG antibody (Sigma).

As expected, MxA was detected in the complex with tubulin, while protein A/G alone did not bind MxA-containing complexes. No binding activity was detected in LOX-pCIneo control cells, further reflecting the specificity of the coimmunoprecipitation.

To further investigate the relationship between MxA and microtubules, soluble proteins were extracted from the LOX cells, using standard techniques, and the insoluble cytoskeletal matrix was examined for associated proteins. Through this analysis, it was determined that only wild-type MxA remained bound to the matrix. T103 mutant WA washed out of the insoluble preparation, indicating that this mutant MxA is soluble and not bound by cytoskeletal elements.

These data support the finding that MxA having an endogenous dynamin/self-assembly region associates with tubulin and the cellular cytoskeleton, whereas mutated MxA does not. More significantly, these data help to establish that microtubules play a role in MxA-mediated regulation of mobility in a unique and unexpected manner.

### EXAMPLE 6

The experiments described in this example further demonstrate that MxA reduces the motility of PC-3M cells.

Using time-lapse video microscopy, PC-3M cells stably transfected with an MxA construct (as described in Example 2) were observed and compared to control PC-3M-β-gal cells. Specifically, PC-3M-βgal and PC-3M-MxA cells were seeded in 25 cm² flasks and cultured for 24 hours. After 24 hours, the flasks were filled with pre-warmed complete medium and cell motility was observed using by phase-contrast microscopy using an Optronics cooled CCD camera mounted on a Leica DMIRB inverted microscope. During observation, the cells were maintained at 37° C. using an ASI 400 Air Stream Incubator (Nevtek—Burnsville, Va.). Time-lapse video microscopy (250 minute recording converted into 1 minute of playing time) was converted into QuickTime movies using Adobe Premier 5.1.

Visual observation confirmed that MxA significantly inhibited the mobility of the PC-3M cells. Additionally, motion pictures of the test and control cells showed active movement of plasma membrane in most cells and several cell divisions, indicating that neither overexpressed MxA, nor β-galactosidase, interfered with mitosis or membrane ruffling.

This experiment serves to corroborate that MA has an impact on mitosis in cells without interfering in mitosis or membrane ruffling. The pronounced decrease in vectorial movement, despite unabated cell membrane activity, observed in these experiments, indicates that MxA targets specific processes regulating motility, such as polarization and/or detachment from substratum adhesion sites (see, e.g., Ballestrem et al., 2000; Wittmann and Wateman-Storer, 2001, for related discussion) and, accordingly, that the invention provides a method of modulating such activities by the administration of an effective amount of WA, MxA homolog, corresponding WA-expressing nucleic acid or vector, or MxA expression-inducing molecule.

### EXAMPLE 7

This example demonstrates the production of a reporter cell suitable for screening potential inducers of MxA promoter activity.

Plasmid pBS-MxA promoter, which contains the MxA promoter sequence (SEQ ID NO:6), was digested with the restriction enzyme Asp718, blunted with Klenow fragment, and further digested with SacI, using standard techniques. Plasmid pGL3 (Promega, Inc.—Madison, Wis.), which contains the firefly luciferase gene (Luc+), was separately digested with SacI and SmaI. The linearized DNAs were separately subjected to agarose gel electrophoresis and the appropriate bands were removed from the gel and purified using standard techniques.

The purified fragments were ligated with T4 DNA ligase (Roche Bioscience—Palo Alto, Calif.), according to regular techniques, to form plasmid pGL3-MxA promoter, which comprises the MxA promoter operably linked in frame to the luciferase gene (SEQ ID NO:7). PC-3-M cells were co-transfected with 6 μg of the MxA construct and 1 μg of pCIneo using Lipofectamine reagent (Invitrogen—Carlsbad, Calif.), according to manufacturer's recommendations. The transfected cells were cultured using standard techniques. The presence of the MxA promoter/luciferase sequence in the transfected cells was confirmed by cycle sequencing. Specifically, both strands were sequenced using BigDye terminator cycle sequencing kit (Applied Biosystems—Foster City, Calif.), using pGL3s1 primer (5′-GCAAGTGCAGGTGCCAGAAC-3′) (SEQ ID NO:8) and PGL3s2 primer (5′-CGTCTTCCATGGTGGCTTTAC-3′) (SEQ ID NO:9).

At 48 hours after transfection, the transfected cells were split at a concentration of 1:15 and plated in selective medium, which contains complete medium and 500 μg/mL of the neomycin analogue G418. The selective medium was replaced every 3 days. After 2 weeks of selection, the plates were inspected for G418-resistant colonies.

Multiple G418-resistant colonies were isolated and isolated colonies were seeded into 6-well plates (in duplicate). At 24 hours after seeding, cells in the plates were treated with either phosphate buffer saline (PBS) as placebo or with 1,000 IU/mL IFN-α to assess the ability of the cell to act as a reporter for MxA promoter induction using a luciferase reporter assay as previously described by Lee et al., J. Biol. Chem. 273:10618-10623 (1998). Briefly, at 20 hours after treatment, the treated cells were washed with PBS and incubated for 20 minutes with reporter lysis buffer (Promega, Inc.). 20 μL of cell lysate was pipetted into each well of a 96-well plate and 100 μL of luciferase assay reagent was added to each well. Emitted light intensity was measured using a Packard luminometer, according to manufacturer's instructions.

Emitted light intensity of control cells was determined and used to provide an average level of induction, Emitted light intensity from eight transfected clones treated with the IFN-α MxA inducer was determined and compared (individually) with the average light intensity from the controls. The results of this experiment are presented in Table 2 as the approximate fold increase in MxA promoter induction (luciferase expression) obtained with treatment with IFN-α as compared to the PBS control.

TABLE 2

| Clone | Fold Induction |
|---|---|
| 1 | 8.1 ± 0.28 |
| 2 | 3.0 ± 0.08 |
| 3 | 4.3 ± 0.43 |
| 4 | 1.6 ± 0.79 |
| 5 | 2.0 ± 0.15 |
| 6 | 5.1 ± 1.14 |
| 7 | 2.0 ± 0.26 |
| 8 | 4.7 ± 0.45 |

The results of these experiments, as set forth in Table 2, reflect that reporter cells incubated with IFN-α, a known inducer of MxA expression, exhibit significantly higher levels of luciferase expression (i.e., at least about 2× and in some cases at least about 4×, at least about 5×, or even at least about 8×) than cells contacted with PBS. More generally, this example demonstrates that cell lines comprising an MxA promoter-reporter gene constructs are able to screen potential inducers of MxA promoter induction in accordance with particular aspects of the invention.

### EXAMPLE 8

This example illustrates particular strategies for identifying compounds that target and upgrade the MxA promoter for developing anti-metastatic and/or anti-cancer therapeutic treatments.

The August 1999 release of the NCI/NIH Developmental Therapeutics Program (DTP) diversity set, comprises 1990 chemotypes selected by defined center analysis of a library of almost 80,000 compounds with the computer program Chem-X (Oxford Molecular Group (now Accelrys)—San Diego, Calif.), described at http://dtp.nci/nih.gov/branches/dscb/diversity %5Fexplanation.html; (see also, Rapisarda et al., Cancer Res., 62(15):4316-4324 (2002), describing the use of the diversity set in the screening of molecules for particular therapeutic uses).

Cells stably transformed with pGL3-MxA are contacted with individual compounds from the DTP diversity set and the cells are monitored for MxA promoter induction by measuring light emission using standard techniques. In an alternative variation of the experiment, one or more selected nucleic acids, such as one or more selected viral RNAs, viral RNA-derived DNAs, viral RNA-derived RNAs (e.g., stability enhanced backbone and/or secondary structure modified RNAs comprising a portion of a viral RNA sequence), or other related nucleic acids (e.g., one or more sequences comprising a viral sequence in combination with unusual base pairs or hybrid RNA/DNA molecules, as described elsewhere herein), are used to screen for MxA promoter induction. In yet another variation, particular cancer-related polypeptides or genes (e.g., selected tumor suppressors) are used to screen for MxA promoter induction. After one or more repetitions of this screening technique, one or more regulators of MxA promoter activity are identified.

This example illustrates strategies by which a MxA promoter-reporter gene construct can be used to screen potential inducers of MxA promoter activity. By employing these and similar strategies, therapeutic agents, such as small molecule inducers of MxA promoter activity, can be identified that can be used for reduction of metastatic potential of cancers and/or the reduction of tumor progression.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Terms such as “including,” “having,” “comprising,” “containing,” and the like are to be construed as open-ended terms (i.e., meaning “including, but not limited to”) unless otherwise indicated, and as encompassing the phrases “consisting of” and “consisting essentially of.” Recitation of ranges of values herein are merely intended to serve as a shorthand method of referring individually to each separate value of the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1.-2. (canceled)

3. A method of assessing the metastatic potential of a cancer comprising:
(a) obtaining a sample of the cancer,
(b) determining the amount of MxA in the sample, amount of MxA-encoding nucleic acid in the sample, level of MxA expression in cells of the sample, or any combination thereof, and
(c) assessing the metastatic potential of the cancer by comparing the amount of MxA in the sample, amount of MxA-encoding nucleic acid in the sample, level of MxA expression in cells of the sample, or any combination thereof with a control or standard.

4.-17. (canceled)

18. The method of claim 3, wherein a decreased amount of MxA in the sample, decreased amount of MxA-encoding nucleic acid in the sample, decreased level of MxA expression in cells of the sample, or any combination thereof as compared to the control or standard is indicative of an increased potential for metastasis, and wherein an increased amount of MxA in the sample, increased amount of MxA-encoding nucleic acid in the sample, increased level of MxA expression in cells of the sample, or any combination thereof as compared to the control or standard is indicative of a decreased potential for metastasis.

19. The method of claim 3, wherein the cancer is selected from the group consisting of prostate cancer, melanoma, breast cancer, colon cancer, and lung cancer.

20. The method of claim 3, wherein the cancer is in a mammal.

21. The method of claim 20, wherein the mammal is a human.

22. The method of claim 20, wherein the amount of MxA in the sample, amount of MxA-encoding nucleic acid, level of MxA expression in cells of the sample, or any combination thereof is lower than the control or standard, and the method further comprises administering an agent to the cancer that reduces the metastatic potential of the cancer.

23. The method of claim 20, wherein the method further comprises prognosticating the likelihood of survival of the mammal.

24. A method of assessing the metastatic potential of a cancer of a host comprising:
(a) obtaining a sample of the cancer, and
(b) assessing the metastatic potential of the cancer by determining the level of expression of MxA having a reduced GTPase activity in cells of the sample, the level of expression of MxA having reduced tubulin association in cells of the sample, or a combination thereof as compared with the level of expression of wild-type MxA in non cancerous cells of the host.

25. The method of claim 24, wherein the MxA having reduced a GTPase activity comprises a mutation in a dynanin GTPase domain.

26. The method of claim 25, wherein the method comprises assaying for an MxA which lacks the N-terminal most threonine residue of the dynamin GTPase domain of the wild-type MxA.

27. The method of claim 24, wherein the method further comprises administering an agent to the cancer that reduces the metastatic potential of the cancer.
